(19) United States
(12) Patent Application Publication
(10) Pub. No.: US 2008/0109058 A1
(43) Pub. Date: May 8, 2008

(54) Intraoperative Anastomosis Method

(75) Inventors: Roy K. Greenberg, Bratenahl, OH (US); Bruce W. Lytle, Bainbridge Township, OH (US); David P. Biggs, Bloomington, IN (US); Lars G. Svensson, Gates Mills, OH (US); Ray Leonard II, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/857,725

(22) Filed: Sep. 19, 2007

Related U.S. Application Data
(63) Continuation-in-part of application No. 11/443,645, filed on May 31, 2006.
(60) Provisional application No. 60/686,656, filed on Jun. 1, 2005.
(60) Provisional application No. 60/845,578, filed on Sep. 19, 2006.

(51) Int. Cl. A61F 2/84; A61F 2/82

(52) U.S. Cl. 623/1.11; 623/1.13

(57) ABSTRACT

An intraoperative anastomosis method is described and comprises the steps of providing an intraluminal prosthesis having a first end and a second end; suturing a first vessel to a second vessel to form a vessel junction; intraluminally delivering the prosthesis so that the first end of the prosthesis is disposed within the first vessel and the second end of the prosthesis is disposed within the second vessel; and deploying the prosthesis so that the prosthesis overlaps and reinforces the vessel junction. The vessel suturing, prosthesis delivering, and prosthesis deploying steps may be performed in a single operation. Other devices, systems, and methods are described.

### RELATED APPLICATIONS

This present patent document is a continuation-in-part of U.S. patent application Ser. No. 11/443,645, filed May 31, 2006, which claims the benefit of the filing date under 35 U.S.C. §119(e) of U.S. Provisional Patent Application Ser. No. 60/686,656, filed Jun. 1, 2005. This present patent document also claims the benefit of the filing date under 35 U.S.C. §119(e) of U.S. Provisional Patent Application Ser. No. 60/845,578, filed Sep. 19, 2006. All of the foregoing applications are hereby incorporated by reference.

### BACKGROUND OF THE INVENTION

1. Field of the Invention

This invention relates to medical devices and, more particularly, to vascular prostheses suitable for various medical applications and the methods for making and using such vascular prostheses.

2. Description of Related Art

Throughout this specification, when discussing the application of this invention to the aorta or other blood vessels, the term “distal” with respect to an abdominal device is intended to refer to a location that is, or a portion of the device that when implanted is, further downstream with respect to blood flow; the term “distally” means in the direction of blood flow or further downstream. The term “proximal” is intended to refer to a location that is, or a portion of the device that when implanted is, further upstream with respect to blood flow; the term “proximally” means in the direction opposite to the direction of blood flow or further upstream.

The functional vessels of human and animal bodies, such as blood vessels and ducts, occasionally weaken or even rupture. For example, the aortic wall can weaken, resulting in an aneurysm. Upon further exposure to hemodynamic forces, such an aneurysm can rupture. In Western European and Australian men who are between 60 and 75 years of age, aortic aneurysms greater than 29 mm in diameter are found in 6.9% of the population, and those greater than 40 mm are present in 1.8% of the population. In particular, aneurysms and dissections that extend into the thoracic aorta and aortic arch are associated with a high morbidity and are, in some situations, particularly difficult to treat.

One intervention for a weakened, aneurismal, dissected or ruptured aorta is the use of an intraluminal device or prosthesis such as a stent graft to provide some or all of the functionality of the original, healthy vessel and/or preserve any remaining vascular integrity by replacing or relining a length of the existing vessel wall that contains the site of vessel weakness or failure. Stent grafts for intraluminal deployment are generally formed from a tube of a biocompatible material in combination with one or more stents to maintain a lumen therethrough. Stent grafts effectively exclude the defect by sealing both proximally and distally to the defect, and shunting blood through its length. A device of this type can, for example, treat various arterial aneurysms, including those in the thoracic aorta or abdominal aorta.

Open surgical (i.e., non-intraluminal) intervention can also be an approach to treating aneurysms or other defects of the aorta. In general, surgical techniques involve repairing the diseased vessel by resecting or physically removing the diseased portion of the vessel. Surgical techniques are typically highly invasive and involve cutting into the body to directly access the diseased or damaged vessel. In the case of an abdominal aortic aneurysm, for example, body organs that obstruct access to the aorta must be repositioned or removed from the body during surgery. The damaged portion of the aorta is cut out and the remaining vessel ends can be anastomosed or joined together to restore vessel function. Alternatively, a tubular graft may be provided and joined between the vessel ends to provide a prosthetic lumen therebetween.

Intraluminal techniques, on the other hand, do not require direct access to the diseased vessel. Instead, an expandable prosthesis is provided and is introduced into the lumen of the vessel, typically through a collateral vessel, remote from the repair site. For example, in the case of an abdominal aortic aneurysm, the prosthesis can be introduced through the femoral artery or the brachial artery. The prosthesis is then delivered to the repair site, whereupon it is expanded into contact with the aorta on either side of the aneurysm, thereby excluding blood flow to the aneurysm.

There are benefits to both intraluminal and non-intraluminal treatments for conditions of the aorta. For example, intraluminal techniques are generally less invasive than surgical techniques, and consequently they are often preferred over surgical techniques. However, not all patients are candidates for intraluminal repair, and so surgical reconstruction is still widely used to repair damaged and diseased body lumens. Hybrid surgical-intraluminal approaches have been described in the literature, including in Greenberg, et al., “Hybrid Approaches to Thoracic Aortic Aneurysms,” 112 Circulation, 2619-2626 (2005) and in Karck, et al., “The frozen elephant trunk technique,” 125 J. Thorac. Cardiovasc. Surg., 1550-3 (2003), both of which are incorporated herein by reference.

One of the challenges associated with surgical resection and reconstruction of a body vessel is providing and ensuring a strong, secure, and lasting anastomosis between vessels. A weak or compromised anastomosis can result in complications that require immediate attention (for example, where the anastomosis leaks) or that are not discovered or discoverable until days, weeks, or even years following the procedure (for example, where the anastomosis weakens over time, resulting in an aneurysm). If a weak or compromised anastomosis is detected during the surgical procedure, it can generally be fixed by oversewing the anastomosis, or by resecting the anastomosis and rejoining the vessels. If a weak or compromised anastomosis is not detected during the surgical procedure, the patient may have to undergo subsequent treatment, resulting in additional time, cost, and risk.

### SUMMARY

An intraoperative anastomosis method is described and comprises the steps of: providing an intraluminal prosthesis having a first end and a second end; suturing a first vessel to a second vessel to form a vessel junction; intraluminally delivering the intraluminal prosthesis so that the first end of the prosthesis is disposed within the first vessel and the second end of the prosthesis is disposed within the second vessel; and deploying the prosthesis so that it overlaps and reinforces the vessel junction. The vessel suturing, prosthesis delivering, and prosthesis deploying steps are preferably performed in a single operation. The vessel suturing step may comprise suturing the first vessel to the second vessel using, for example, sutures, staples, or the like. The intraluminal prosthesis may comprise, for example, a stent or a stent graft.

In preferred methods, an intraluminal prosthesis may be delivered and deployed to overlap and reinforce a vessel junction, as a prophylactic measure, rather than a remedial measure. Thus, in some examples, the intraluminal prosthesis is used proactively (i.e., to prevent occurrence of damage to the vessels and vessel junction), rather than reactively (i.e., to repair damage to the vessels and the vessel junction).

At least one of the first vessel and the second vessel may be a prosthetic vessel, such as a graft or a stent graft, or a body vessel, such as the aorta, esophagus, trachea, ureter, bile duct, and the like. In some examples, the first and second vessel may each be a body vessel. In other examples, the first and second vessel may each be a prosthetic vessel. In further examples, one of the first vessel and the second vessel may be a prosthetic vessel and the other of the second vessel and the first vessel may be a body vessel.

Intraluminal access for the prosthesis may be provided through a vessel that is proximate the vessel junction. For example, where one of the vessels is a prosthetic vessel, intraluminal access may be provided through an incision in the prosthetic vessel. Likewise, where one of the vessels is a body vessel, intraluminal access for the prosthesis may be provided through an incision in the body vessel. Alternatively, intraluminal access may be provided through an incision in a vessel that is remote from the first and second vessels. For example, where one of the vessels is the aorta, intraluminal access may be provided through a femoral or brachial artery.

In some examples, the intraluminal prosthesis may comprise a stent graft comprising a graft and at least one stent. The prosthesis may further comprise at least one hook or barb extending from the at least one stent. In a preferred example, a stent graft may comprise at least one Z-stent that is disposed on an inside surface and/or on an outside surface of the graft. In some examples, the stent graft may comprise at least one fenestration or scallop.

The deploying step may comprise deploying a stent graft so that at least one stent overlaps the vessel junction. An advantage of this feature is that the stent may provide radial support to the vessel junction and may limit or prevent compression, twisting, kinking, or other deformation which could damage or deteriorate the vessel junction, the first vessel, and/or the second vessel.

In an exemplary method, the first vessel comprises the aorta and the second vessel comprises a tapered tubular graft having a distal end and a proximal end. The method may include one or more of the steps described above. In addition, the method may include one or more of the steps of: placing a distal portion of the tapered tubular graft inside the aorta, and suturing the proximal end of the graft in place. The step of suturing the proximal end of the graft in place may comprise, for example, suturing the proximal end of the graft to the aorta.

Other methods may include one or more additional steps, such as: providing at least one stent attached to the tapered tubular graft at a site adjacent the distal end of the graft; loading the tapered tubular graft into an introducer; inserting the introducer through an incision in the aorta; and deploying the tapered tubular graft inside the aorta.

In some examples, the tapered tubular graft may comprise a fenestration and a method may comprise resecting an island from the aorta and suturing the island to the fenestration.

Intraluminal access for the tapered graft may be provided, for example, through an incision in the aorta. Intraluminal access for the intraluminal prosthesis may be provided, for example, through an incision in the aorta or an incision in the tapered tubular graft. In other examples, intraluminal access for the tapered graft and/or the intraluminal prosthesis may be provided through an incision in another vessel that is remote from the aorta and the tapered tubular graft.

Another intraoperative anastomosis method is described and comprises the steps of providing an intraluminal prosthesis; intraluminally delivering and deploying the intraluminal prosthesis within a first vessel so that a first end of the intraluminal prosthesis is disposed adjacent an opening in the first vessel; and joining the first vessel to a second vessel by suturing the first end of the intraluminal prosthesis to an opening in the second vessel.

The intraluminal prosthesis may comprise, for example, a stent graft comprising a graft and at least one stent attached to the graft. The prosthesis may further comprise at least one hook or barb extending from the at least one stent. In some examples, the intraluminal prosthesis may comprise a suture ring disposed adjacent the first end of the graft and the suturing step may comprise the step of suturing the second vessel opening to the suture ring. An exemplary method may be used to repair an acute dissection of the distal ascending aorta, where the first vessel comprises the distal ascending aorta and the second vessel comprises a second graft.

### BRIEF DESCRIPTION OF THE DRAWINGS

FIG. 1 shows a stent graft having stents at the distal end;

FIG. 2 shows the stent graft of FIG. 1 with the addition of a scallop at the proximal end;

FIG. 3a shows a stent graft sutured at its proximal end to a preexisting graft;

FIG. 3b shows an island sutured to a graft that extends into the ascending aorta;

FIG. 4a shows a stent graft similar to that of FIG. 1, having an uncovered stent at its distal end;

FIG. 4b shows a shorter version of the graft of FIG. 4a;

FIG. 5a shows a detailed view of a stent graft with stents at its distal end;

FIG. 5b shows an internal view of the stent graft of FIG. 5a;

FIG. 6 shows a variation of the stent graft of FIG. 5a;

FIG. 7 shows a sealing cuff;

FIGS. 8-15 show various views of a first introducer in different stages of deployment;

FIGS. 16-17 show a second exemplary introducer;

FIGS. 18-19 show stent grafts disposed at vessel junctions;

FIGS. 20-26 show various stages of an exemplary intraoperative anastomosis method;

FIG. 27 shows an aorta that has been repaired using an exemplary intraoperative anastomosis method;

FIGS. 28-30 show various stages of another exemplary intraoperative anastomosis method; and

FIGS. 31A and 31B show intraluminal prostheses that may be used, for example, in an intraoperative anastomosis method.

### DETAILED DESCRIPTION

To help understand this description, the following definitions are provided.

The term “anastomosis” refers to any existing or established connection between two lumens, such as the prosthetic trunk and prosthetic branch, that puts the two in fluid communication with each other. An anastomosis is not limited to a surgical connection between blood vessels, and includes an integrally formed connection between a prosthetic branch and a prosthetic trunk.

The term “intraoperative” means occurring within an operation. The term “operation” means a process or series of acts involved to perform a particular function or achieve a particular result.

The term “prosthesis” means any replacement for a body part or function of that body part. It can also mean a device that enhances or adds functionality to a physiological system.

The term “lumen” describes a cavity or channel within a tube or a tubular body, such as vessel. A lumen can be an existing lumen or a lumen created by surgical intervention. This includes lumens, such as blood vessels, parts of the gastrointestinal tract, ducts such as bile ducts, parts of the respiratory system, etc. The term “intraluminal” means within a lumen, and describes objects that are found or that can be placed inside a lumen in the human or animal body, or methods or processes that occur within a lumen. An “intraluminal prosthesis” is thus a prosthesis that is found or that can be placed within a lumen. A stent graft is a type of intraluminal prosthesis that has a graft component and a stent component.

The term “stent” means any device or structure that adds rigidity, expansion force or support to a prosthesis. A “Z-stent” is a stent that has alternating struts and peaks (i.e., bends) and defines a generally cylindrical space.

The term “expandable” means capable of being expanded. An expandable stent is a stent that is capable of being expanded, whether by virtue of its own resilience, upon the application of an external force, or by a combination of both. Expandable stents may be self-expanding and/or balloon expandable. Self-expanding stents can be made of stainless steel, materials with elastic memory properties, such as NITINOL, or any other suitable material. Exemplary self-expanding stents include Z-STENTS® and ZILVER® stents, which are available from Cook Incorporated, Bloomington, Ind., USA. Balloon expandable stents may be made, for example, of stainless steel (typically 316LSS, CoCr, etc.). Hybrid stents may be provided, for example, by combining one or more self-expanding stents or stent portions with one or more balloon expandable stents or stent portions.

The term “vessel” refers to a tube or canal in which fluid may be contained and conveyed or circulated. A body vessel (as opposed to a prosthetic vessel) is a vessel that naturally exists, or is naturally formed in the body. Examples of body vessels include, but are not limited to, blood vessels such as the aorta and the femoral artery, the esophagus, the trachea, the ureter, the bile duct, etc. Examples of prosthetic vessels include grafts and stent grafts.

The term “graft” describes an object, device, or structure that is joined or that is capable of being joined to a body part to enhance, repair, or replace a portion or a function of that body part. Grafts that can be used to repair body vessels include, for example, films, coatings, or sheets of material that are formed or adapted to conform to the body vessel that is being enhanced, repaired, or replaced. A stent may be attached to a graft to form a “stent graft.”

Biocompatible fabrics, non-woven materials and porous sheets may be used as the graft material. The graft material is preferably a woven polyester having a twill weave and a porosity of about 350 ml/min/cm² (available from VASCUTEK® Ltd., Renfrewshire, Scotland, UK). The graft material may also be other polyester fabrics, polytetrafluoroethylene (PTFE), expanded PTFE, and other synthetic materials known to those of skill in the art.

The graft material may include extracellular matrix materials. The “extracellular matrix” is a collagen-rich substance that is found in between cells in animal tissue and serves as a structural element in tissues. It is typically a complex mixture of polysaccharides and proteins secreted by cells. The extracellular matrix can be isolated and treated in a variety of ways. Following isolation and treatment, it is referred to as an “extracellular matrix material,” or ECMM. ECMMs may be isolated from submucosa (including small intestine submucosa), stomach submucosa, urinary bladder submucosa, tissue mucosa, renal capsule, dura mater, liver basement membrane, pericardium or other tissues.

Purified tela submucosa, a preferred type of ECMM, has been previously described in U.S. Pat. Nos. 6,206,931; 6,358,284 and 6,666,892 as a bio-compatible, non-thrombogenic material that enhances the repair of damaged or diseased host tissues. U.S. Pat. Nos. 6,206,931; 6,358,284 and 6,666,892 are incorporated herein by reference. Purified submucosa extracted from the small intestine (“small intestine submucosa” or “SIS”) is a more preferred type of ECMM for use in this invention. Another type of ECMM, isolated from liver basement membrane, is described in U.S. Pat. No. 6,379,710, which is incorporated herein by reference. ECMM may also be isolated from pericardium, as described in U.S. Pat. No. 4,502,159, which is also incorporated herein by reference. Other examples of ECMMs are stomach submucosa, liver basement membrane, urinary bladder submucosa, tissue mucosa and dura mater. SIS can be made in the fashion described in U.S. Pat. No. 4,902,508 to Badylak et al.; U.S. Pat. No. 5,733,337 to Carr; U.S. Pat. No. 6,206,931 to Cook et al.; U.S. Pat. No. 6,358,284 to Fearnot et al.; 17 Nature Biotechnology 1083 (November 1999); and WIPO Publication WO 98/22158 of May 28, 1998 to Cook et al., which is the published application of PCT/US97/14855; all of these references are incorporated herein by reference. It is also preferable that the material is non-porous so that it does not leak or sweat under physiologic forces.

Biocompatible polyurethanes may also be employed as graft materials. One example of a biocompatible polyurethane is THORALON (THORATEC, Pleasanton, Calif.), as described in U.S. Pat. Nos. 6,939,377 and 4,675,361, both of which are incorporated herein by reference. THORALON is a polyurethane base polymer (referred to as BPS-215) blended with a siloxane-containing surface-modifying additive (referred to as SMA-300). The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be manipulated to provide either porous or non-porous THORALON. Porous THORALON can be formed by mixing the polyetherurethane urea (BPS-215), the surface modifying additive (SMA-300) and a particulate substance in a solvent. The particulate may be any of a variety of different particulates or pore forming agents, including inorganic salts. Preferably the particulate is insoluble in the solvent. The solvent may include dimethyl formamide (DMF), tetrahydrofuran (THF), dimethyacetamide (DMAC), dimethyl sulfoxide (DMSO) or mixtures thereof. The composition can contain from about 5 wt % to about 40 wt % polymer, and different levels of polymer within the range can be used to fine tune the viscosity needed for a given process. The composition can contain less than 5 wt % polymer for some spray application embodiments. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water for the extraction, for example, water at a temperature of about 60° C. The resulting pore diameter can also be substantially equal to the diameter of the salt grains.

The porous polymeric sheet can have a void-to-volume ratio from about 0.40 to about 0.90. Preferably the void-to-volume ratio is from about 0.65 to about 0.80. The resulting void-to-volume ratio can be substantially equal to the ratio of salt volume to the volume of the polymer plus the salt. Void-to-volume ratio is defined as the volume of the pores divided by the total volume of the polymeric layer including the volume of the pores. The void-to-volume ratio can be measured using the protocol described in AAMI (Association for the Advancement of Medical Instrumentation) VP20-1994, Cardiovascular Implants—Vascular Prosthesis section 8.2.1.2, Method for Gravimetric Determination of Porosity. The pores in the polymer can have an average pore diameter from about 1 micron to about 400 microns. Preferably, the average pore diameter is from about 1 micron to about 100 microns; more preferably, it is from about 1 micron to about 10 microns. The average pore diameter is measured based on images from a scanning electron microscope (SEM). Formation of porous THORALON is described, for example, in U.S. Pat. No. 6,752,826 and US. Patent Application Publication No. 2003/0149471 A1, both of which are incorporated herein by reference.

Non-porous THORALON can be formed by mixing the polyetherurethane urea (BPS-21 5) and the surface modifying additive (SMA-300) in a solvent, such as dimethyl formamide (DMF), tetrahydrofuran (THF), dimethyacetamide (DMAC) or dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes may also be employed. These include polyurethanes that preferably include a soft segment and include a hard segment formed from a diisocyanate and diamine. For example, polyurethane with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

The diisocyanate used as a component of the hard segment may be represented by the formula OCN—R—NCO, where —R— may be aliphatic, aromatic, cycloaliphatic or a mixture of aliphatic and aromatic moieties. Examples of diisocyanates include MDI, tetramethylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof.

Biocompatible polyurethanes modified with cationic, anionic and aliphatic side chains may also be used, as in U.S. Pat. No. 5,017,664.

Other biocompatible polyurethanes include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif., USA).

Other biocompatible polyurethanes include polyurethanes having siloxane segments, also referred to as a siloxane-polyurethane. Examples of polyurethanes containing siloxane segments include polyether siloxanepolyurethanes, polycarbonate siloxane-polyurethanes, and siloxanepolyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxanepolyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL, the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Patent Application Publication No. 2002/0187288 A1, which is incorporated herein by reference.

In addition, any of these biocompatible polyurethanes may be end-capped with surface active end groups, such as, for example, polydimethylsiloxane, fluoropolymers, polyolefin, polyethylene oxide or other suitable groups. See, for example, the surface active end groups disclosed in U.S. Pat. No. 5,589,563, which is incorporated herein by reference.

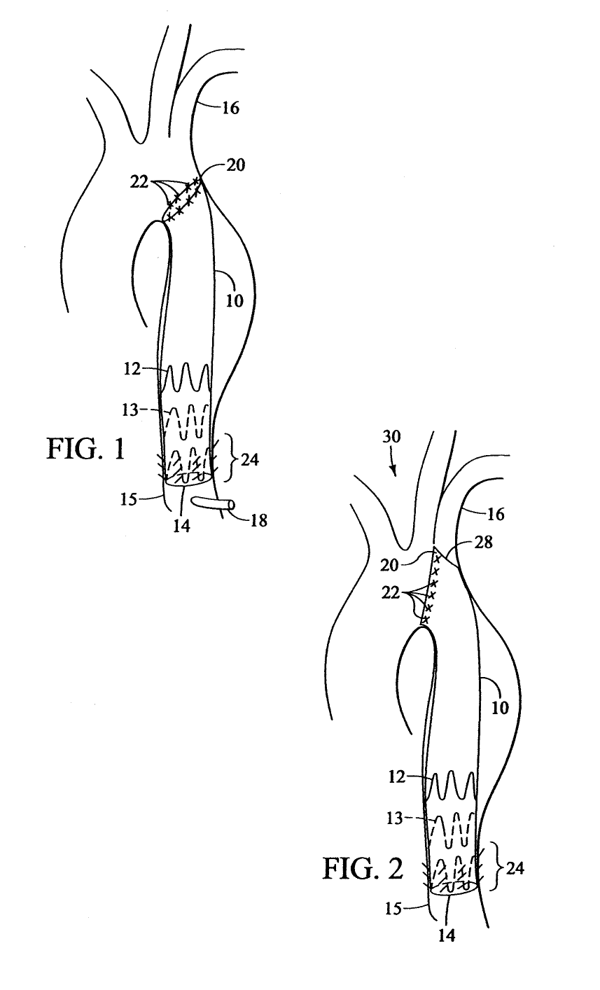

FIG. 1 shows a stent graft 10 designed for implantation in the thoracic aorta. The stent graft is formed from a section of graft material shaped into a tube. Stents 12 are positioned at the distal end 14 of the graft 10. The graft tube may be made of any of the graft materials described above, preferably woven polyester twill. The fabric may be crimped so that the graft may be able to bend without excessive kinking. The graft tube is preferably sized to correspond to a particular patient's anatomy. An exemplary graft 10 may have a length of about 140-280 mm, and may be designed to extend from a point just distal of the subclavian artery 16 to a point proximal to the celiac artery 18, as shown in FIG. 1. The stent graft 10 may be manufactured to a maximum length, and be subsequently trimmed to suit a particular patient. The diameter of an exemplary graft 10 is about 30 mm. The graft 10 is preferably tapered. For example, the proximal end 20 of the graft 10 may have a diameter of 30 mm, while the distal end 14 of the graft 10 may have a diameter of 28, 32, 36 or 44 mm. Thus, the graft 10 either tapers distally (i.e., is narrower in the distal region of the graft 10), or tapers proximally (i.e., is narrower in the proximal region of the graft 10). The taper can better allow the stent graft 10 to form a sealing interconnection with preexisting grafts.

The proximal end 20 of the stent graft 10 is preferably unstented, as it is designed to be anastomosed to the native artery with sutures 22, as shown in FIG. 1 and described in further detail below. The distal end 14, however, is preferably stented so that a seal may be formed between the stent graft 10 and the native artery following deployment of the stent graft 10, without the addition of sutures. As used herein, the terms “suture,” “suturing,” and the like, shall include other similar or equivalent anastomosis devices and techniques including, for example, staples and stapling techniques.

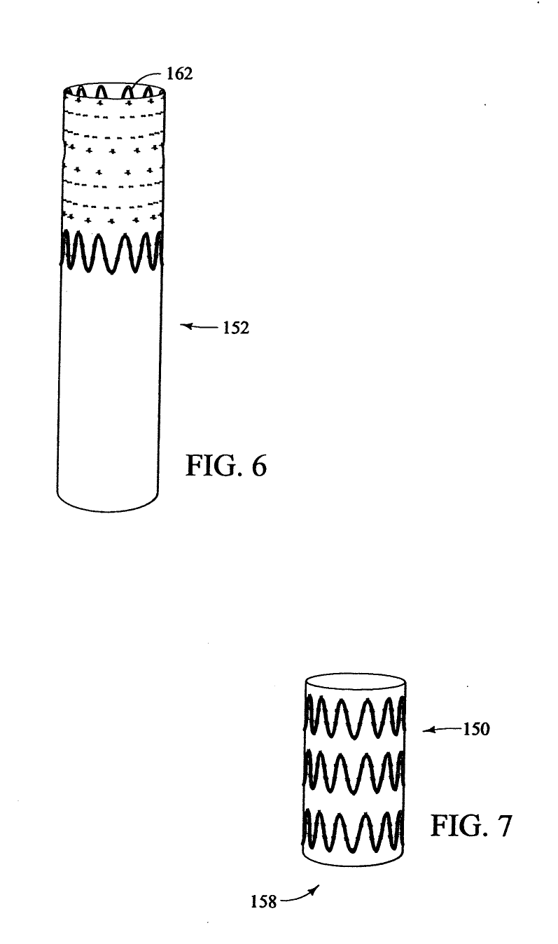

As shown in FIG. 1, there are preferably three self-expanding Z-stents 12, 13, 15 sutured to the distal end 14 of the graft 10. The distal stent 15 is preferably sutured to the inside of the graft 10, as shown in FIG. 1. This may improve the circumferential apposition of the stent graft 10 to the surrounding vessel wall. The other stents 12, 13 can be sutured to the outside of the graft 10. Alternatively, two distal-most stents may be sutured to the inside of the graft and the third stent can be sutured to the outside of the stent graft 10, as shown at the distal end 162 of the graft 152 in FIG. 6. Thus, in an embodiment that has three Z-stents, the stents can have an approximate amplitude of 17.5 mm, such that the three of them, sutured to the distal end of the graft, occupy about a 60 mm length of the graft. The remainder of the graft can be about 80-85 mm, for a total length of about 140-145 mm. There may be more stents added to the distal end, depending on the overall length of the graft, the requirements of the anatomy, etc.

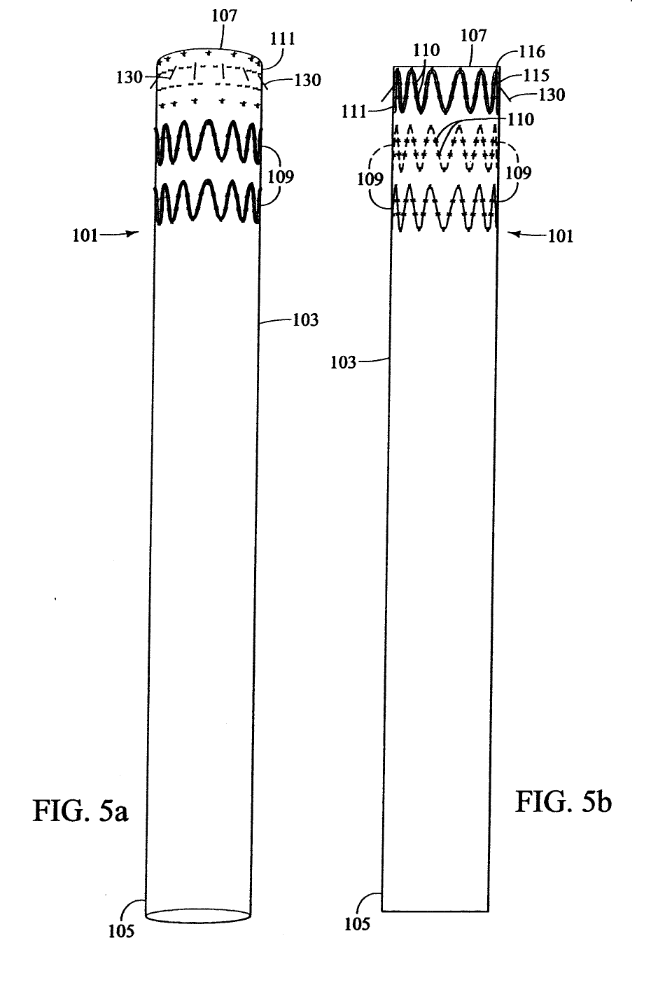

Barbs or hooks 24 preferably extend in the proximal (cephalad) direction from the distal-most stent 15. Barbs 24 may also extend from the other stents 12, 13. The barbs 24 may help anchor the distal end 14 of the graft 10 in place, thereby improving sealing at the distal end 14. The barbs may also provide traction so that during an operation, the surgeon may pull the graft tight to decrease slack or wrinkles in the graft prior to anastomosing the graft to the native artery. The barbs 24 may extend from the struts or the bends of the Z-stent 15. There may be a single ring of barbs 24 extending from the stent 15, or a more extensive array of barbs as shown in FIG. 1. A single row of barbs 130 extending from the distal bends of a Z-stent is shown in FIG. 5.

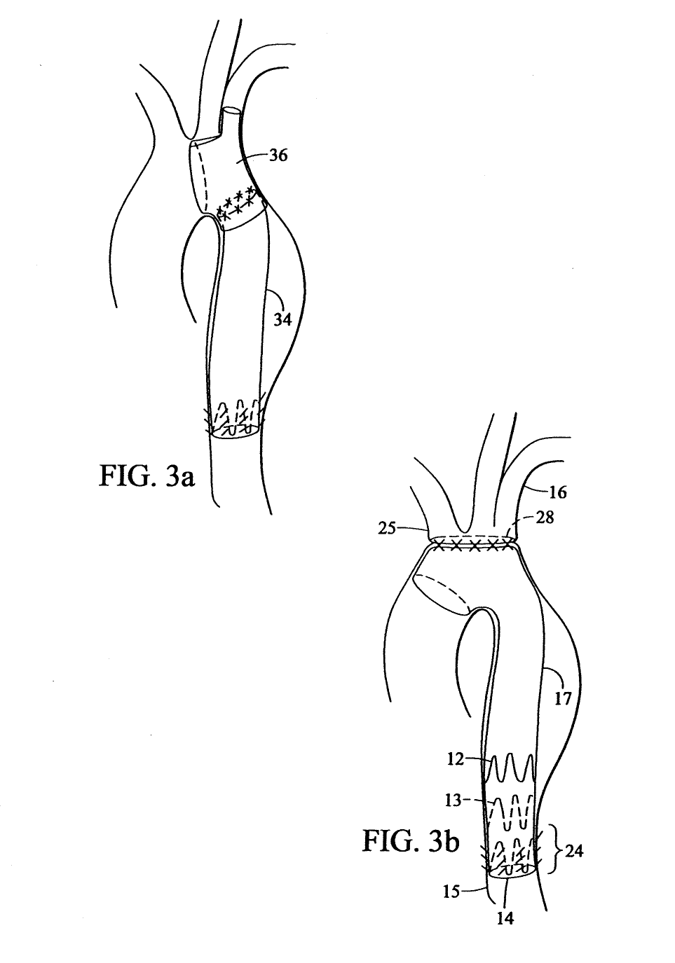

As shown in FIG. 2, the proximal end 20 of the stent graft 10 may have a scallop 28 that accommodates the subclavian artery 16, allowing the stent graft 10 to be sutured at a more proximal location in the aortic arch 30, while not impeding flow to the subclavian artery 16. A proximal fenestration or an integral prosthetic branch (not shown) may also be employed for a similar purpose.

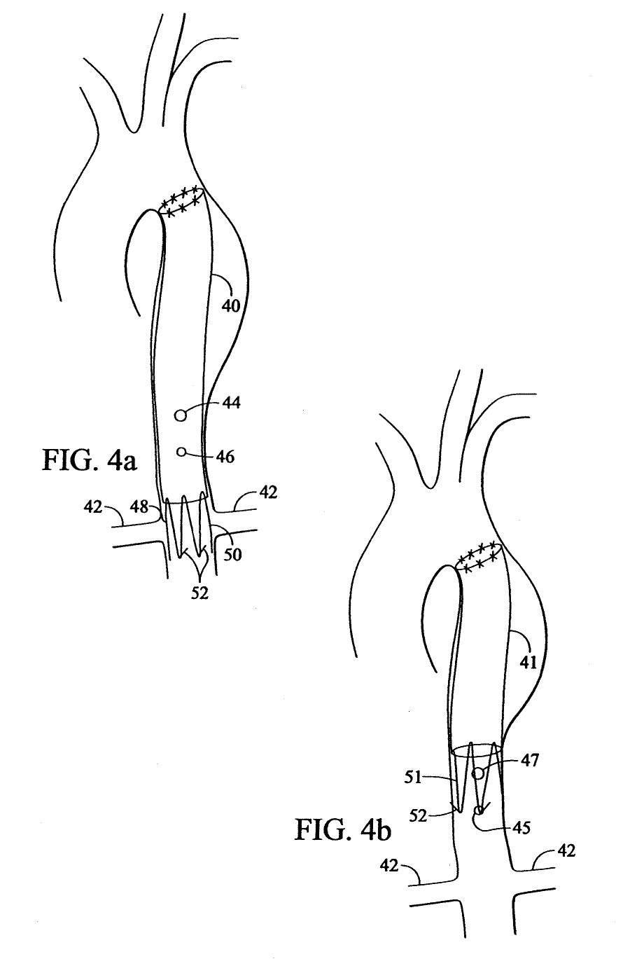

An extension for an integral prosthetic branch may be deployed. As shown in FIG. 3a, the stent graft 34 may be sutured to a preexisting prosthetic module 36. The preexisting prosthetic module 36 may have been deployed surgically or intraluminally during the same operation or in a previous operation.

The graft can also extend further proximally with the use of an open surgical procedure using the “island” surgical technique. In that technique, the aorta is clamped proximally to the innominate, left common carotid, and left subclavian arteries. An island 25 encompassing those aortic arch side branches is cut from the aorta. A graft 17 having a fenestration 27 that approximates the shape and size of the island is deployed into the aortic arch. Alternatively, the fenestration 27 can be cut after the graft's deployment. The island 25 is then sutured to the fenestration 27 and the location in the aorta from which the island was resected.

FIG. 4a shows that the distal end 52 of the graft 40 may be modified to accommodate the branch vessels of the thoraco-abdominal aorta, such as the celiac, SMA and renal arteries. As with the subclavian, these can be accommodated with, for example, fenestrations, scallops, or integral prosthetic branches. FIG. 4a shows a stent graft 40 extending to the renal arteries 42. A celiac fenestration 44 and SMA fenestration 46 preserve blood flow to their respective arteries. The distal end 48 of the stent graft 40 features an uncovered stent 50 that extends over the renal arteries 42 without occluding them. Barbs 52 extend proximally from the uncovered stent 50.

FIG. 4b shows a shorter graft than that of FIG. 4a. In FIG. 4b, the uncovered stent transverses the SMA 45 and celiac 47 arteries so that they are not occluded.

FIGS. 5a and 5b show external and internal views of an embodiment of an exemplary stent graft. The stent graft 101 includes a tubular body 103 formed from a biocompatible woven or non-woven fabric, or other material. The tubular body 103 has a proximal end 105 and a distal end 107. The stent graft 101 may be tapered, as described above, depending upon the topography of the vasculature and flow considerations.

Towards the distal end 107 of the tubular body 103, there are a number of self-expanding Z-stents 109, 111 such as the Z-stent on the outside of the body. In this embodiment there are two external stents 109 spaced apart by a distance of between 0 mm to 10 mm. The external stents 109 are joined to the graft material by means of stitching or suturing 110, preferably using a monofilament or braided suture material.

At the distal end 107 of the prosthetic module 101 there is provided an internal Z-stent 111 which provides a sealing function for the distal end 107 of the stent graft 101. The outer surface of the tubular body 103 at the distal end 107 presents an essentially smooth outer surface, which, with the assistance of the internal Z-stent 111, can engage and seal against the wall of the aorta when it expands and is deployed.

The internal stent 111 is comprised of struts 115 with bends 116 at each end of the struts. Affixed to some or all of the struts 115 are barbs 130 which extend proximally from the struts 115 through the graft material. When the stent graft is deployed into an aortic arch, the barbs 130 engage and/or penetrate into the wall of the aorta and prevent proximal movement of the stent graft 101 caused by pulsating blood flow through the stent graft 101. It will be noted that the stent 111 is joined to the graft material by means of stitching 112, preferably using a monofilament or braided suture material.

FIG. 6 shows a stent graft 152 that has the two distal stents attached to the inside 162 of the stent graft 152. Additional stent grafts are described in U.S. Patent Application Publication Nos. 2003/0199967 A1 and 2004/0106978 A1, which are incorporated herein by reference.

FIG. 7 shows a three-stent cuff 150 that can be used in conjunction with the stent grafts described above. The cuff 150 may be about 55 mm in length and can have any of a variety of suitable diameters including, for example, 28, 30 or 32 mm. It is preferably sized to form a sealing interconnection with a stent graft. The stents 158 may be placed internally and/or externally, relative to the main graft. It can be introduced with a 30 cm variation of one of the introducers described below. Once the stent graft described below is deployed, the cuff shown in FIG. 7 may be used to seal the distal or proximal end of the stent graft if it becomes apparent that the stent graft itself does not exhibit optimal sealing against the aortic wall. In particular, the sealing cuff may be used at the site of surgical anastomosis (i.e., the proximal portion of the graft), if it is discovered that the surgical anastomosis exhibits imperfect sealing. By placing a sealing cuff using intraluminal techniques, surgical repair of the anastomosis may be rendered unnecessary. Such a cuff 150 may also be manufactured using two or four stents, for example.

The devices described above may be implanted using a hybrid surgical procedure—one that employs aspects of open surgical repair in addition to intraluminal techniques. In summary, the aortic arch is surgically exposed; then an incision is made in the aortic arch or associated branch vessel so that an introducer containing the stent graft can be inserted into the aorta. The aortic arch can be exposed using a conventional median sternotomy. The introducer is advanced distally through the aortic arch into the thoracic aorta, until it is in a proper distal position. At that point, the stent graft is released from the introducer. At the distal end of the stent graft, the stents expand, with or without the assistance of a balloon catheter, thereby forming a seal at the distal end. Then, the proximal end of the stent graft—which is preferably stent-free—is sutured to the native aorta using standard surgical techniques. Finally, the incision in the aortic arch is closed, followed by the closure of the surgical access.

Thus, using this hybrid procedure, a second surgical operation through a separate entry point—e.g., a left thoracotomy—is rendered unnecessary to ensure sealing at the distal end of the stent graft.

Exemplary introducers are described further below.

Introducer

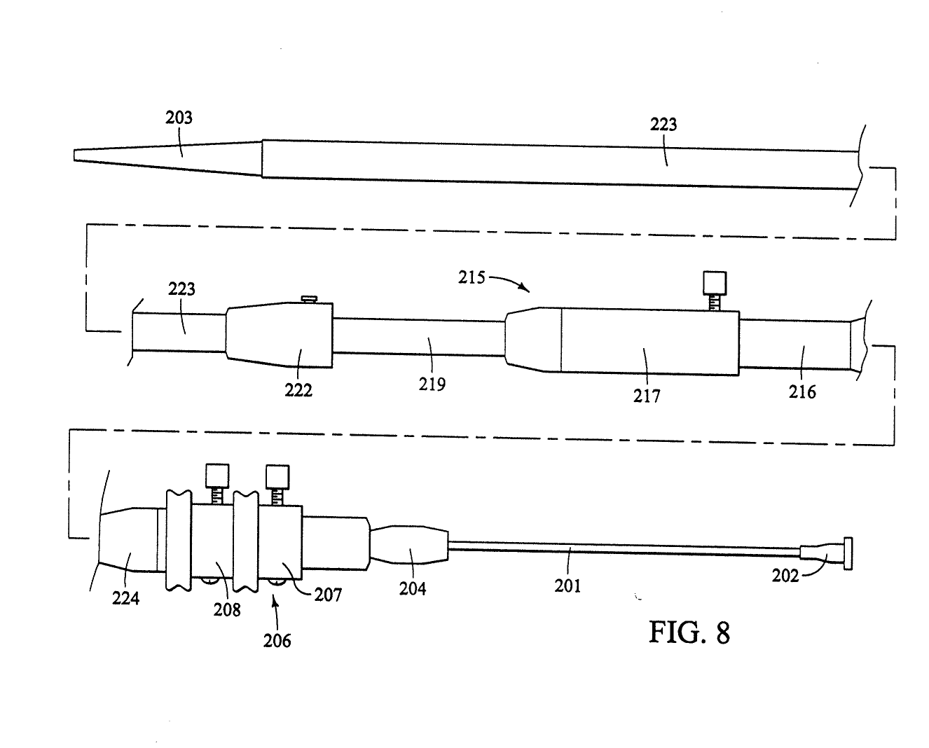

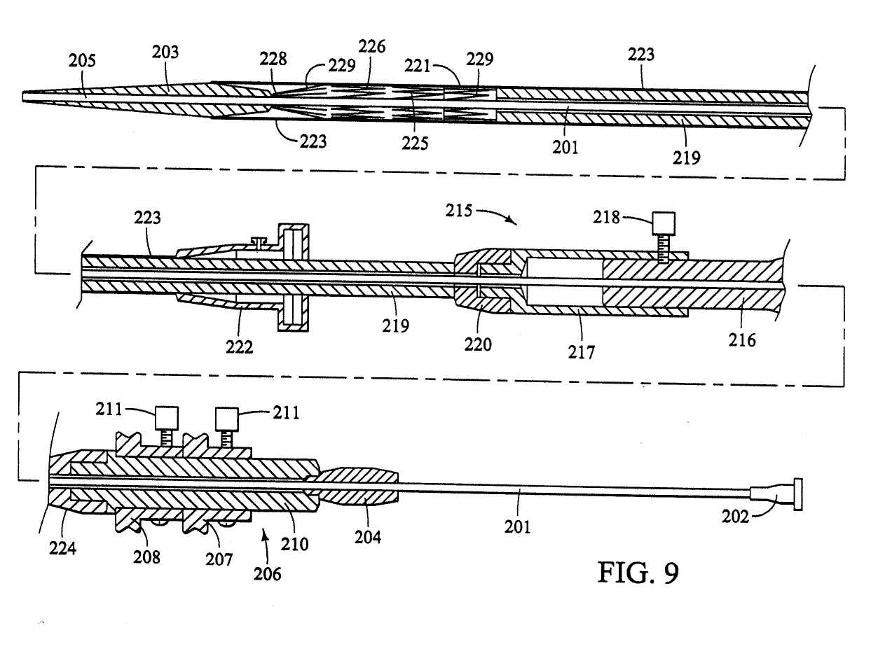

FIGS. 8 and 9 show an exemplary introducer which may be used to deploy the stent graft described above. The introducer may be about 40 cm in length, which is shorter than the delivery systems that are used to deploy stent grafts through femoral cut-downs. For example, the TX-2 delivery system (Cook Incorporated, Bloomington, Ind.) is generally about 75 cm. The introducer, shown in FIGS. 8 and 9, is preferably about 20/22 French in diameter.

The introducer may comprise, working from the inside towards the outside, a guide wire catheter 201 which extends the full length of the device from a syringe socket 202 at the far distal end of the introducer to a nose dilator 203 at the proximal end of the introducer. The introducer may also be employed without the assistance of a guide wire, and thus will lack a guide wire catheter and associated features.

The nose cone dilator 203 is fixed to the guide wire catheter 201 and moves with it; the dilator may be about 40 mm and is preferably blunt tipped. The nose cone dilator has a through bore 205 as an extension of the lumen of the guide wire catheter 201 so that the introducer can be deployed over a guide wire (not shown). To lock the guide wire catheter 201 with respect to the introducer in general, a pin vice 204 is provided. Again, a version of the introducer shown in FIGS. 8 and 9 may be designed so that it works without a guide wire, and thus, does not have the bore 205 and other features used with a guide wire.

The trigger wire release mechanism generally shown as 206 at the distal end of the introducer includes a distal end trigger wire release mechanism 207 and a proximal end trigger wire release mechanism 208. The trigger wire release mechanisms 207 and 208 slide on a portion of the fixed handle 210. Until such time as they are activated, the trigger wire mechanisms 207 and 208 are fixed by thumbscrews 211 (FIG. 9) and remain fixed with respect to the fixed portion of the fixed handle. The controlled deployment afforded by use of the trigger wires helps to ensure accurate placement of the distal portion of the graft.

Immediately proximal of the trigger wire release mechanism 206 is a sliding handle mechanism generally shown as 215. The sliding handle mechanism 215 generally includes a fixed handle extension 216 of the fixed handle 210 and a sliding portion 217. The sliding portion 217 slides over the fixed handle extension 216. A thumbscrew 218 fixes the sliding portion 217 with respect to the fixed portion 216. The fixed handle portion 216 is affixed to the trigger wire mechanism handle 210 by a screw threaded nut 224. The sliding portion of the handle 217 is fixed to the deployment catheter 219 by a mounting nut 220. A deployment catheter extends from the sliding handle 217 through to a capsule 221 at the proximal end of the deployment catheter 219.

Over the deployment catheter 219 is a sheath manipulator 222 and a sheath 223, which slide with respect to the deployment catheter 219 and, in the ready to deploy situation as shown in FIGS. 8 and 9, extend from the sheath manipulator 222 forward to the nose cone dilator 203 to cover a prosthetic module 225 retained on the introducer distally of the nose cone dilator 203.

In the ready to deploy condition shown in FIGS. 8 and 9, the sheath 223 assists in retaining stent graft 225, which includes self-expanding stents 226 in a compressed condition. The proximal covered stent 227 is retained by a fastening at 228 which is locked by a trigger wire (not shown) which extends to trigger wire release mechanism 208. The distal exposed stent 229 on the stent graft 225 is retained within the capsule 221 on the deployment catheter 219 and is prevented from being released from the capsule by a distal trigger wire (not shown), which extends to the distal trigger wire release mechanism 207.

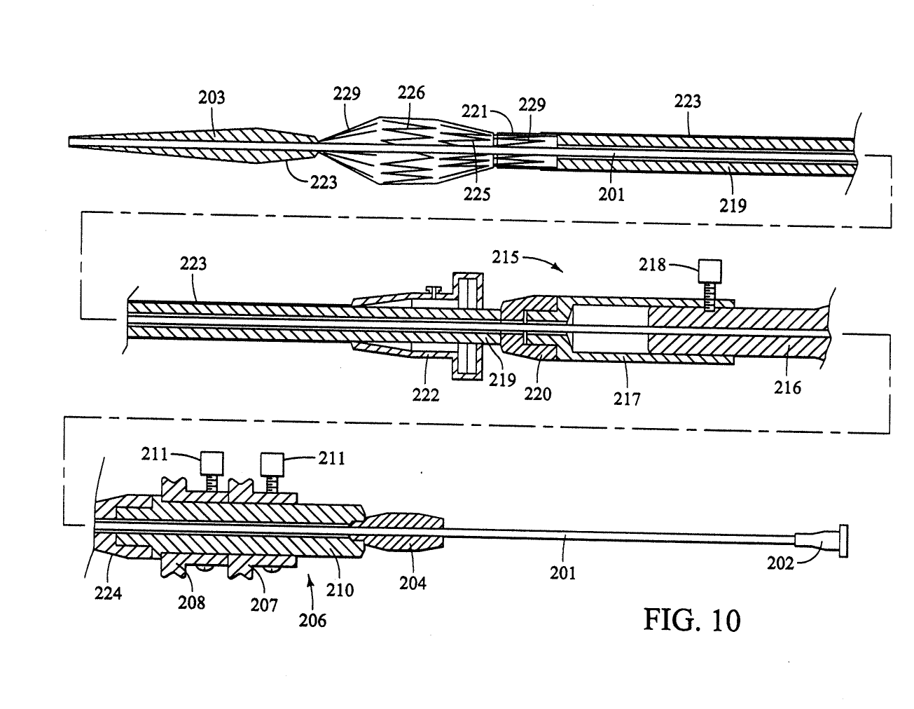

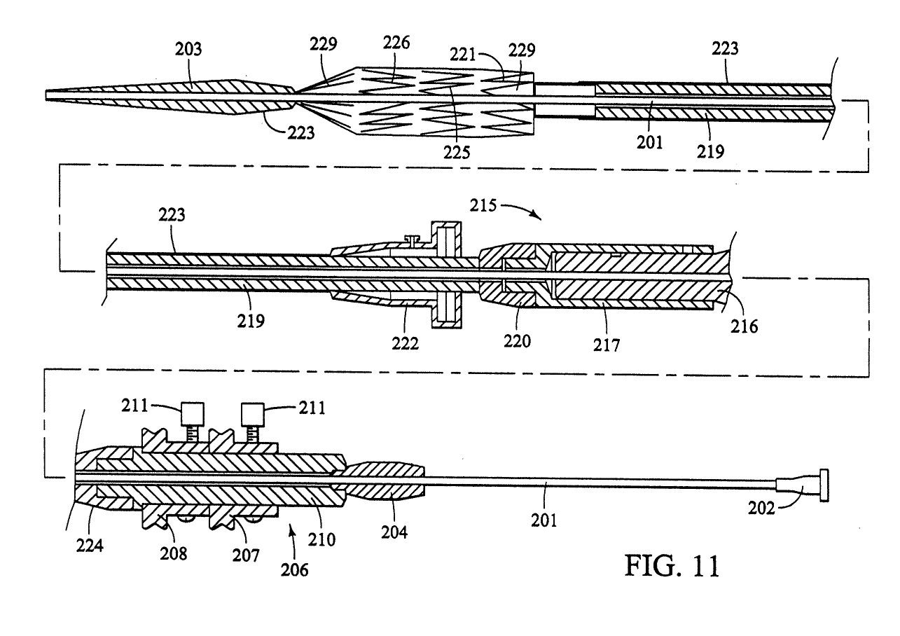

FIG. 9 shows the same view as FIG. 8, but after withdrawal of sheath 223, and FIG. 11 shows the same view as FIG. 10, but after activation of sliding handle mechanism 215.

In FIG. 10, the sheath manipulator 222 has been moved distally so that its proximal end clears the stent graft 225 and lies over the capsule 221. Freed of constraint, the self-expanding stents 226 of the stent graft 225 are able to expand. However, the fastening 228 still retains the uncovered stent 229, and the capsule 221 still retains the other stents. At this stage, the proximal and distal ends of the stent graft 225 can be independently repositioned, although if the distal stent 229 included barbs as it has in some embodiments, the proximal end can only be moved proximally.

Once repositioning has been done, the distal end of the stent graft 225 should be released first. The distal trigger wire release mechanism 207 on the handle 210 is removed to withdraw the distal trigger wire. Then the thumb screw 218 is removed, and the sliding handle 217 is moved distally to the position shown in FIG. 11. This moves the capsule 221 to release the exposed stent 229. As the fastening 228 is retained on the guide wire catheter 201, just distal of the nose cone dilator 203, and the guide wire catheter 201 is locked in position on the handle 210 by pin vice 204, then the proximal trigger wire release mechanism 208, which is on the handle 210, does not move when moving the sliding handle, deployment catheter 219 and capsule 221, so the proximal end of the prosthetic module 225 remains in a retained position. The proximal end of the prosthetic module 225 can be again manipulated at this stage by manipulation of the handle. Although, if the uncovered stent 229 included barbs as discussed above, the proximal end can only be moved proximally. The proximal fastening 228 can then be released by removal of the proximal trigger wire release mechanism 208.

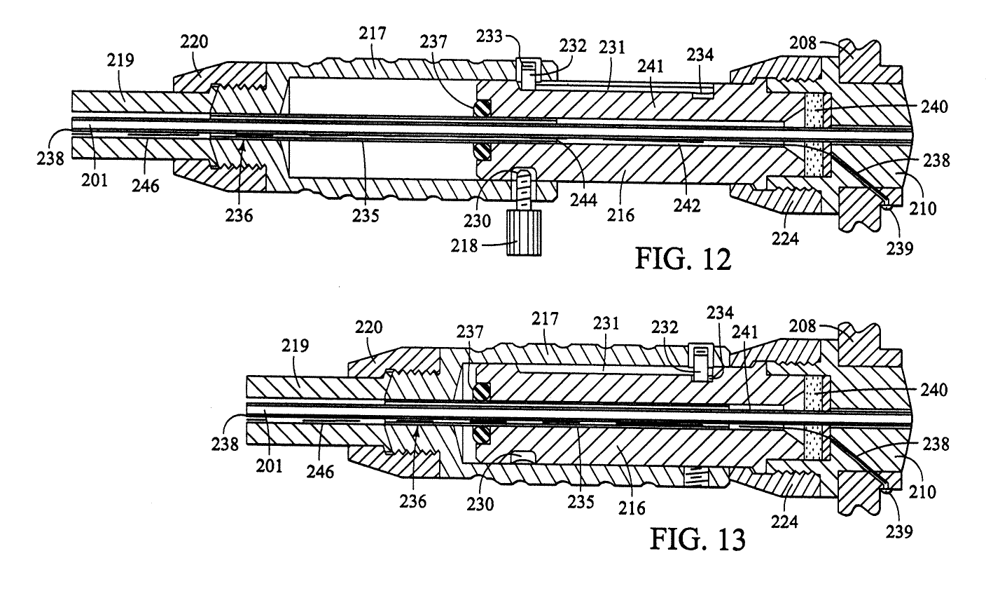

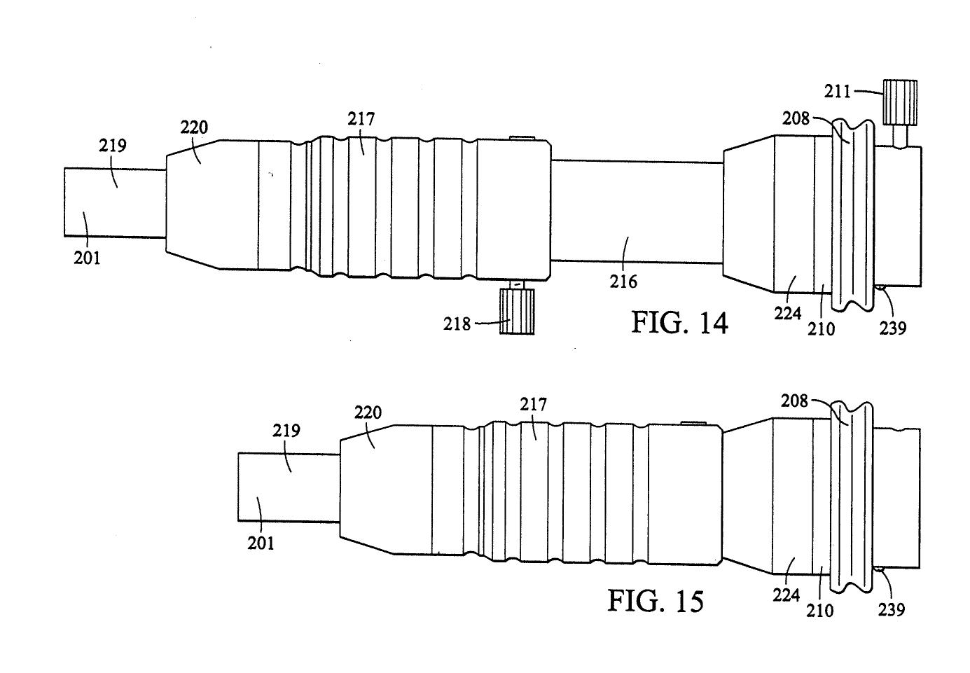

As shown in FIGS. 12 and 13, the detailed construction of a particular embodiment of a sliding handle mechanism according to this invention is shown. FIGS. 12 and 14 show the sliding handle mechanism in the ready to deploy condition. FIGS. 13 and 15 show the mechanism when the deployment catheter and hence the capsule has been withdrawn by moving the sliding handle with respect to the fixed handle. The fixed handle extension 216 is joined to the trigger wire mechanism handle 210 by screw threaded nut 224.

The sliding handle 217 is fixed to the deployment catheter 219 by screw threaded fixing nut 220 so that the deployment catheter moves along with the sliding handle 217. The sliding handle 217 fits over the fixed handle extension 216 and, in the ready to deploy situation, is fixed in relation to the fixed handle by locking thumbscrew 218, which engages into a recess 230 in the fixed handle extension 216. On the opposite side of the fixed handle extension 216 is a longitudinal track 231 into which a plunger pin 232 spring loaded by means of spring 233 is engaged. At the distal end of the track 231 is a recess 234.

A guide tube 235 is fixed into the proximal end of the sliding handle 217 at 236 and extends back to engage into a central lumen 241 in the fixed handle extension 216 but is able to move in the central lumen 241. An O ring 237 seals between the fixed handle extension 216 and guide tube 235. This provides a hemostatic seal for the sliding handle mechanism. The trigger wire 238, which is fixed to the trigger wire releasing mechanism 208 by means of screw 239, passes through the annular recess 242 between the fixed handle extension 216 and the guide wire catheter 201 and then more proximally in the annular recess 244 between the guide wire catheter 201 and the guide tube 235 and forward to extend through the annular recess 246 between the guide wire catheter 201 and the deployment catheter 219 and continues forward to the proximal retaining arrangement. Similarly, the distal trigger wire (not shown) extends to the distal retaining arrangement.

A further hemostatic seal 240 is provided where the guide wire catheter 201 enters the trigger wire mechanism handle 210 and the trigger wires 238 pass through the hemostatic seal 240 to ensure a good blood seal.

As can be seen in FIGS. 13 and 15, the locking thumbscrew 218 has been removed and discarded, and as the sliding handle is moved onto the fixed handle, the plunger pin 232 has slid back along the track 231 to engage into the recess 234. At this stage, the sliding handle cannot be moved forward again.

As the trigger wire release mechanisms 207 and 208 are on the trigger wire mechanism handle 210, which is fixed with respect to the fixed handle 216, then the proximal trigger wire 238 is not moved when the deployment catheter 219 and the sliding handle 217 are moved so that it remains in position and does not prematurely disengage.

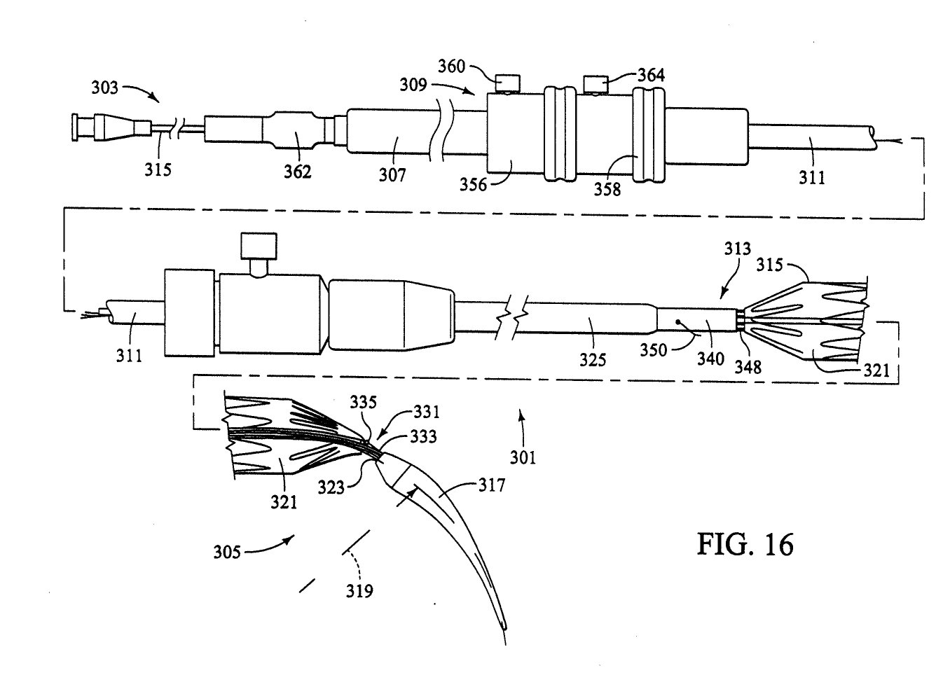

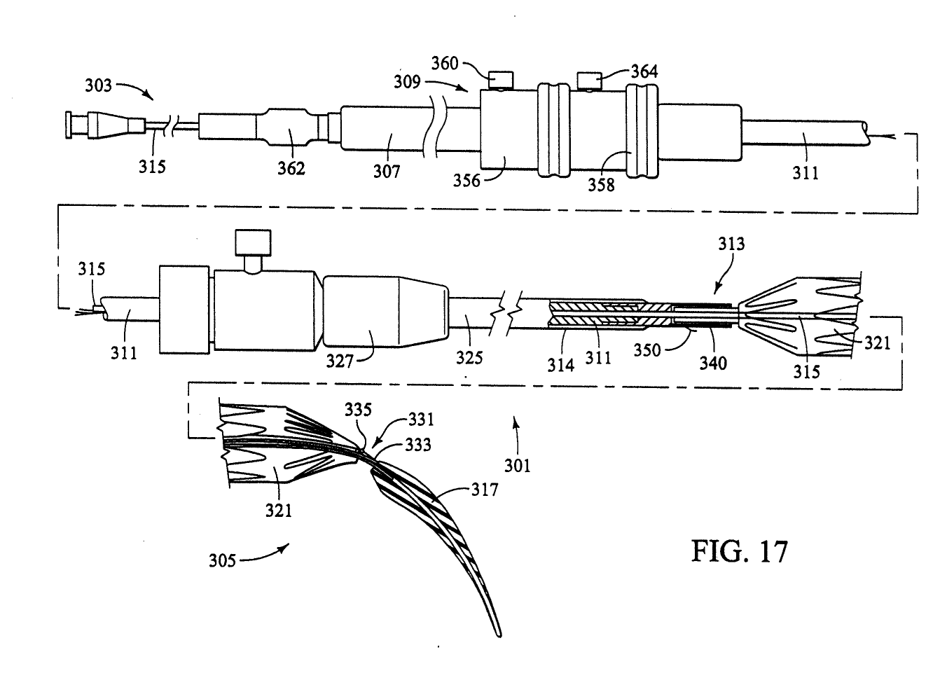

FIGS. 16 and 17 show an alternative introducer 301 that has a distal end 303 which in use is intended to remain outside a patient and a proximal end 305 which is introduced into the patient. This introducer is further described in U.S. Patent Application Publication No. 2004/0106974, which is incorporated herein by reference. The curved nose cone dilator 317 may help guide the introducer 301 through the aortic arch or tortuous anatomy.

Towards the distal end there is a handle arrangement 307 which includes trigger wire release apparatus 309 as will be discussed later. The main body of the introducer includes a tubular carrier 311 which extends from the handle 307 to a proximal retention arrangement, generally shown as 313.

Within a longitudinal lumen 314 in the central carrier 311 extends a guide wire catheter 315. The guide wire catheter 315 extends out through the proximal retention arrangement 313 and extends to a nose cone dilator 317 at the distal end of the introducer 301. The nose cone dilator 317 is curved, and in the embodiment shown in FIG. 39, the guide wire catheter 315 is also curved towards its distal end so that the distal end 305 of the introducer has a curve which may have a radius of curvature 319 of between 70 to 150 mm. This curvature enables the introducer of the present invention to be introduced through the aortic arch of a patient without excessive load being placed on the walls of the aorta.

A stent graft 321 is retained on the introducer between the distal end 323 of the nose cone dilator 317 and the distal retention arrangement 313. A sleeve 325 fits over the tubular carrier 311, and, by operation of a sleeve manipulator 327, the sleeve can be extended forward to extend to the nose cone dilator 317. By the use of the sleeve 325, the stent graft 321 can be held in a constrained position within the sleeve.

At the distal end of the stent graft just proximal of the proximal end 323 of the nose cone dilator 317, a distal retention arrangement 331 is provided.

The distal retention arrangement 331 includes a trigger wire 333, which engages a knot 335 of suture material, which is fastened to the trigger wire 333 and the guide wire catheter 315. When the trigger wire 333 is withdrawn as will be discussed later, the suture knot 325 is released and the distal end of the stent graft can be released. The nose cone dilator 317 can have one or more apertures extending longitudinally, and the proximal trigger wire 333 can extend into one of these apertures.

The proximal retention arrangement 313, as shown in detail in FIG. 40, includes a capsule 340, which is part of a capsule assembly 341, which is joined by a screw thread 343 to the distal end 342 of the central carrier 311. The capsule 340 includes a passageway 344 within it with a proximal closed end 346 and an open distal end 348. The open distal end 348 faces the nose cone dilator 317 and the guide wire catheter 315 passes through the center of passageway 344.

The stent graft 321 has a distal stent 348 that is received within the capsule 340, which holds it constrained during deployment. If the distal stent 348 has barbs extending from its struts, then the capsule keeps the barbs from prematurely engaging the walls of the vessel it is being deployed in and also prevents them from catching in the sleeve 325. A trigger wire 350 passes through aperture 352 in the side of the capsule, engages a loop of the exposed stent 348 within the capsule and then passes along the annular recess 354 between the guide wire catheter 315 and the tubular carrier 311 to the trigger wire release mechanism 309.

The trigger wire release mechanism 309 includes a proximal release mechanism 356 and a distal end release mechanism 358.

To release the stent graft after it has been placed in the desired position in the aorta, the sleeve 325 is withdrawn by pulling back on the sleeve manipulator 327 while holding the handle 307 stationary. The distal release mechanism 358 on the handle 307 is then released by loosening the thumb screw 364 and completely withdrawing the distal release mechanism 358, which pulls out the trigger wire 333 from the capsule 340. Pin vice 362, which fixes the position of the guide wire catheter with aspect to the handle 307 and central carrier 311, is then loosened so that the guide wire catheter 315 can be held stationary, which holds the nose cone dilator and hence the distal retention arrangement 331 stationary while the handle is pulled back to remove the capsule 340 from the exposed stent 348, which releases the distal end of the stent graft.

Once the position of the distal end of the stent graft 321 has been checked, the proximal release mechanism 358 can then be removed by release of the thumb screw 364 and complete removal of the proximal release mechanism 358.

The tubular central carrier 311 can then be advanced while holding the nose cone dilator 317 stationary so that the introducer can be made more compact for withdrawal. Then the proximal end of the stent graft can be sutured in place, as described above.

Various other introducers or delivery and deployment devices may be used and are described, for example, in PCT Application WO 98/53761 entitled “A Prosthesis and a Method and Means of Deploying a Prosthesis,” United States Published Patent Application No. 2003/0233140 entitled “Trigger Wire System,” and United States Published Patent Application No. 2004/0098079 entitled “Thoracic Aortic Stent Graft Deployment Device,” the disclosures of which are herein incorporated by reference.

Intraoperative Anastomosis Methods

As described above, intraluminal devices may be implanted using hybrid or intraoperative methods that comprise aspects of open surgical repair and aspects of intraluminal repair. For example, an intraluminal prosthesis may be provided and used at the site of a surgical anastomosis to reinforce the connection between the anastomosed vessels.

In some examples, two vessels may be surgically joined, and an intraluminal prosthesis may be delivered and deployed to form an overlapping interconnection with the two vessels. The intraluminal prosthesis may comprise, for example, a stent or a stent graft. Suitable stents and graft materials are described above. Suitable stent grafts are described above and include, for example, the prosthetic cuff shown in FIG. 7. The intraluminal prosthesis may be used remedially, for example, to repair a surgical anastomosis or junction if it becomes apparent that the anastomosis exhibits imperfect sealing. In some preferred examples, the intraluminal prosthesis may be used prophylactically, rather than remedially, to protect the vessels and anastomosis and to prevent damage or deterioration to the anastomosis that may otherwise result in an unprotected vessel.

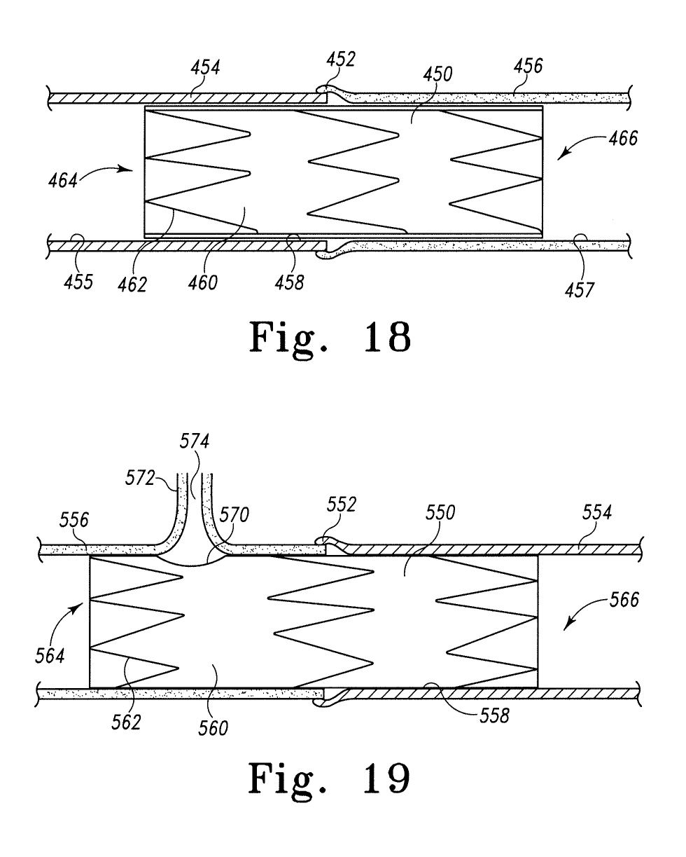

FIG. 18 depicts a first vessel 454 and a second vessel 456 that are joined at an anastomosis or vessel junction 452. The vessels 454, 456 may be surgically joined by a suturing technique, as described and defined above. The first vessel 454 has a lumen 455 and may comprise, for example, a prosthetic vessel or a body vessel. The second vessel 456 has a lumen 457 and may comprise, for example, a prosthetic vessel or a body vessel. The first vessel 454 and the second vessel 456 define a common flow lumen 458.

In some examples, the first and second vessels may each comprise prosthetic vessels, as shown in FIG. 3A. In other examples, the first and second vessels may each comprise body vessels. In the example shown in FIG. 18, the first vessel 454 is a prosthetic vessel and the second vessel 456 is a body vessel.

Once the two vessels 454, 456 are joined, the surgeon may test the quality of the vessel junction 452. If the junction 452 is weak or compromised, for example if there is a leak, the junction may be repaired using a traditional surgical technique such as oversewing or resection and rejoinder. Once the surgeon is satisfied with the surgical anastomosis, an intraluminal prosthesis 450, may be provided to overlap and reinforce the vessel junction 452.

In the example shown in FIG. 18, a stent graft 450 is provided and comprises a generally tubular graft 460 and at least one expandable stent 462. The stent graft 450 is sized according to the dimensions of the common lumen 458. For example, for applications within the aortic arch or the descending aorta of a human adult, the expanded diameter of the stent graft 50 will preferably be 25 mm or greater. Other suitable sizes include 28 mm, 30 mm, and 32 mm. The length of the stent graft 450 will be sufficient to overlap the junction 452 and to effectively seal the junction 452 on either side. The inventors have discovered that a length of approximately 55 mm is preferred, although shorter or longer stent grafts may be used where appropriate and are contemplated within the scope of the invention.

The stent graft 450 preferably comprises two or more self-expanding Z-stents 462. Each of the stents 462 is preferably disposed on the inner surface of the graft 460, however, some or all of the stents 462 may be disposed on the exterior surface of the graft 460. The stents 462 may be attached to the graft 460 by various mechanisms, such as by suture or adhesive, and/or by incorporating or interweaving the stent into the graft material. The stent graft 450 may comprise barbs or hooks (not shown) that may help anchor the stent graft in place. In one example, the stent graft 450 may comprise barbs or hooks on a proximally-disposed stent and on a distally-disposed stent to help anchor and seal the stent graft proximally and distally of the vessel junction 452. Examples of suitable stent grafts and configurations thereof are described in PCT Application WO 98/53761, previously incorporated by reference.

The stent graft 450 is placed within the common flow lumen 458 and has a first end 464 that is disposed within the first vessel 454 and a second end 466 that is disposed within the second vessel 456. The stent graft 450 overlaps the vessel junction 452 and forms an overlapping interconnection with the first vessel 454 and the second vessel 456. The stent graft 450 is positioned so that the graft material 460 overlaps, reinforces, and seals the junction 452. Accordingly, the stent graft 450 protects the junction 452 from fluid pressure that could weaken and erode the vessel junction. At least one of the stents 462 is preferably positioned in an overlapping relationship with the junction 452 to provide radial support thereto.

FIG. 19 depicts another exemplary stent graft 550 which is placed in a common flow lumen 558 between a first vessel 554 and a second vessel 556. The stent graft 550 comprises a graft 560 and one or more stents 562, as described above. The vessels are anastomosed and a vessel junction 552 is provided between the first and second vessels 554, 556, adjacent a branch vessel 572 that extends from the second vessel 556. A first end 564 of the stent graft 550 is disposed within the second vessel 556 and the second end 566 of the stent graft is disposed within the first vessel 554. The stent graft 550 overlaps the vessel junction 552 and is positioned within the common flow lumen 558 so that it does not occlude the branch vessel lumen 574. The stent graft 550 may include one or more apertures or fenestrations 570 and/or one or more scallops (not shown) formed in the graft material, that may be coordinated with the ostia of the branch vessel 572. During delivery and deployment of the stent graft 550, the fenestrations and/or scallops are positioned so that the stent graft 550 does not cover or occlude adjacent branch vessel lumens.

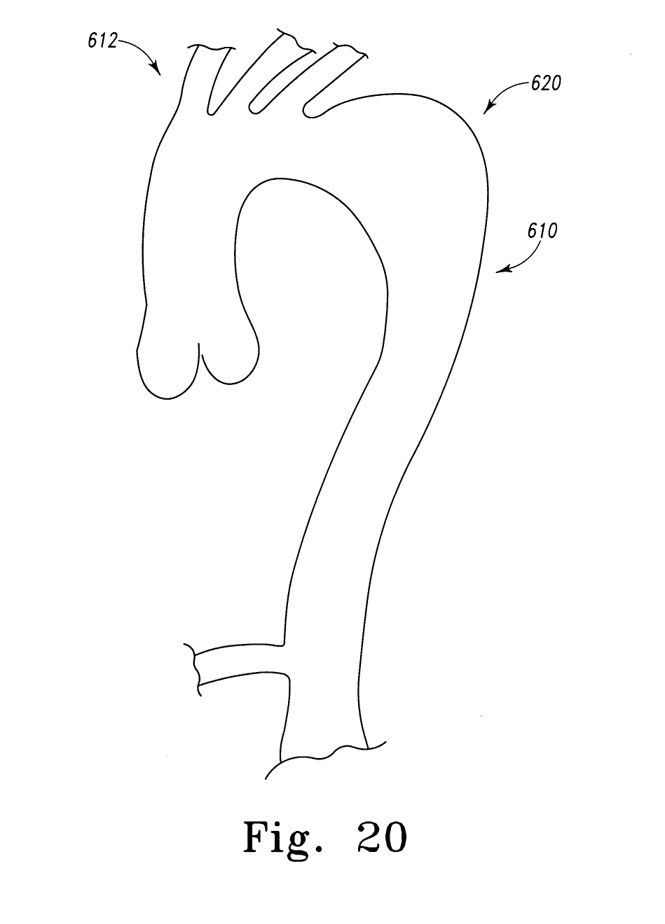

An exemplary intraoperative method will now be described with reference to FIGS. 20-26. FIG. 20 shows a schematic view of a region of the aorta 610 including the aortic arch 612. An aneurysm 620 is formed in the aorta 610 and includes the aortic arch 612.

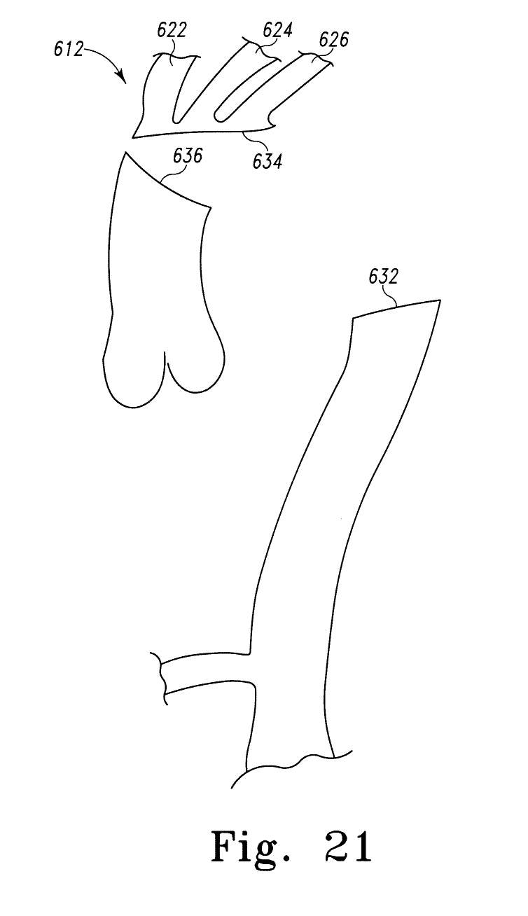

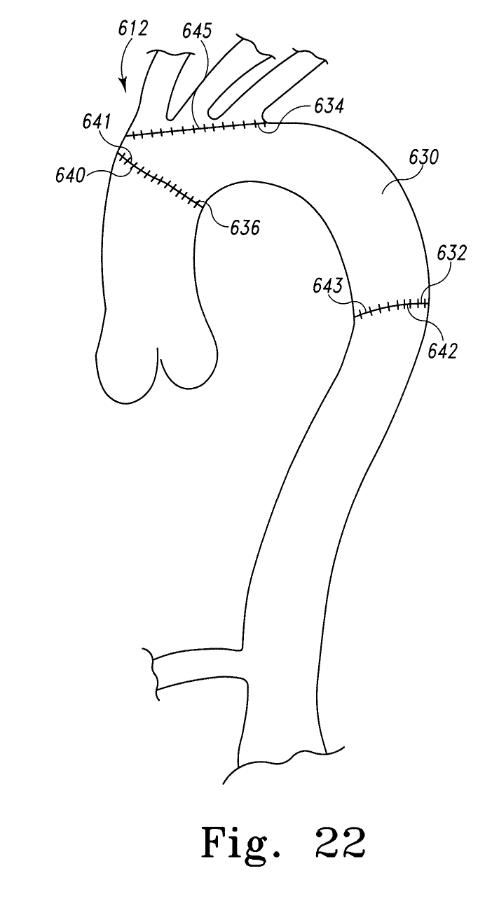

FIG. 21 shows the aorta 610 of FIG. 20, wherein the aneurysm 620 has been resected or removed. To perform a resection, the surgeon first cools the patient, and then exposes the aorta through a sternotomy. The surgeon then performs cardiopulmonary bypass or systemic circulatory arrest. Next, the aneurysm 620 is cut out or resected. In the example shown in FIG. 21, the resection exposes a proximal end opening 632 in the descending aorta, a proximal end opening 634 adjacent branch vessels 622, 624, 626, and a distal end opening 636 in the ascending aorta.

The aortic arch 612 may be reconstructed, for example, using a graft and/or a stent graft and a surgical or hybrid technique. Exemplary techniques are described above and are depicted in FIGS. 1-4. In the example shown in FIG. 22, the aortic arch 612 is reconstructed using a generally tubular graft 630. The proximal end 640 of the graft 630 is joined to distal end opening 636 to form vessel junction 641. The distal end 642 of the graft 630 is joined to proximal end opening 632 to form vessel junction 643. An opening is provided in the wall of the graft 630 that corresponds with proximal end opening 634. The graft 630 is joined to proximal end opening 634 to form vessel junction 645. The junctions 641, 643, 645 may be secured by suturing. Alternative securing devices or techniques will be readily recognized by one of ordinary skill in the art and are contemplated within the scope of the present invention.

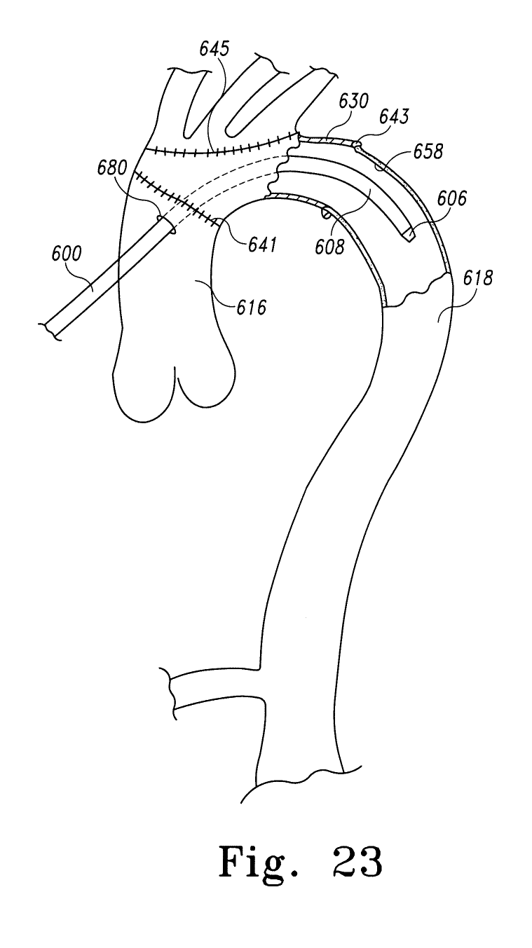

In FIG. 23, an incision 680 is made in a vessel to provide intraluminal access for delivering and deploying a stent graft (not shown). As shown, the incision 680 may be made proximate the vessel junction, for example, in the ascending aorta 616. Access could be provided through an incision in other proximate locations, such as the graft 630, or in the descending aorta 618. In other examples, an incision may be provided in a remote or collateral vessel, such as the femoral or brachial arteries. The stent graft is inserted through the incision 680 via an introducer 600, such as one of the introducers described above and depicted in FIGS. 8-17. The stent graft is delivered to the common lumen 658 and is positioned adjacent anastomosis 643, as shown in FIG. 23.

If remote access is provided for delivering and deploying the stent graft, the introducer 600 and/or the stent graft may be imageable so that the position of the stent graft may be monitored during the procedure. For example, the stent graft and/or the introducer may comprise radiopaque indicia or markers for fluoroscopic viewing. If, on the other hand, access is provided locally, or proximate the vessel junction, the operator may be able to directly view the positioning of the stent graft during the procedure, and therefore such imaging devices and techniques may not be required.

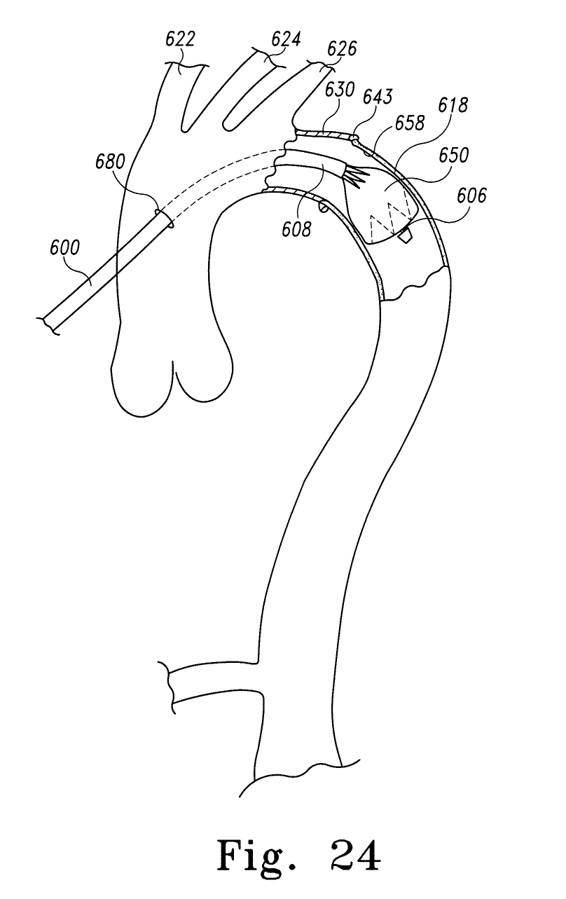

Once the stent graft is properly positioned, the introducer sheath 608 may be retracted to expose the stent graft and to release it from the sheath 608. In FIG. 24, a distal portion of the stent graft 650 is expanded within the common lumen 658 and is positioned distal to the vessel junction 643 within the descending aorta 618. At this point, the proximal end of the stent graft 650 is still retained within the sheath 608 and the stent graft 650 may be lengthened or shortened or otherwise moved for accurate positioning. The stent graft 650 may also be recaptured at this stage by simply sliding the sheath towards the proximal end of the introducer 600.

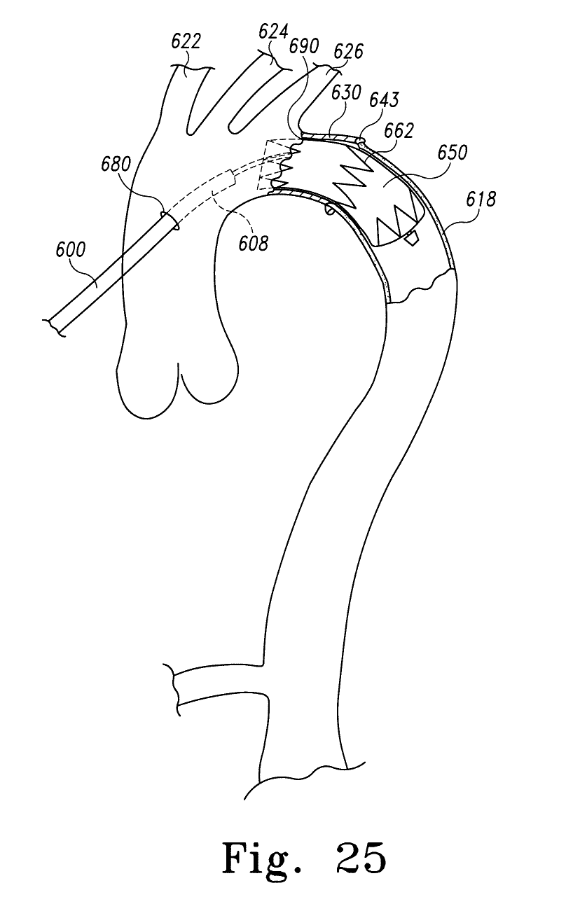

In FIG. 25, the sheath 608 is completely removed from the stent graft 650. The stent graft 650 is expanded so that the proximal end of the stent graft 650 is disposed within graft 630 and the distal end is disposed within the descending aorta 618. The stent graft 650 forms an overlapping interconnection with the graft 630 and the descending aorta 618. A scallop 690 is provided at the proximal end of the stent graft 650 so that the stent graft can be placed without occluding blood flow to the left subclavian artery 626.

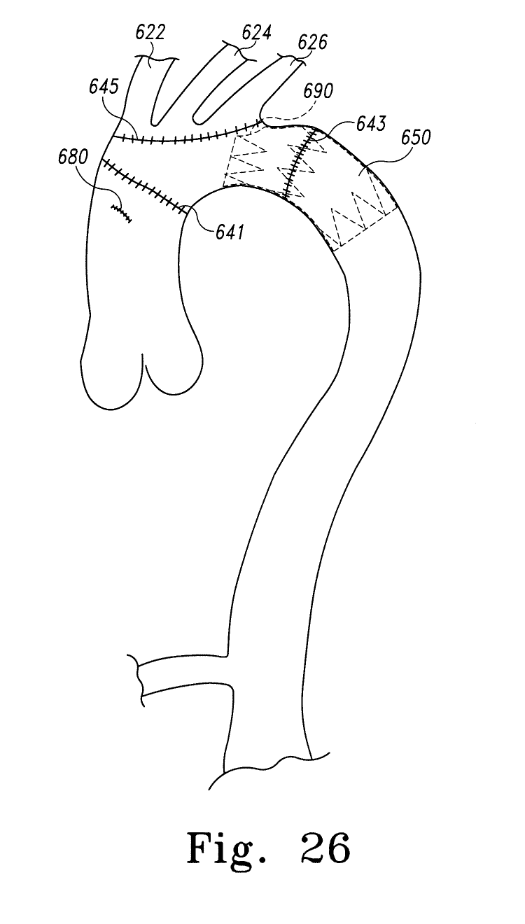

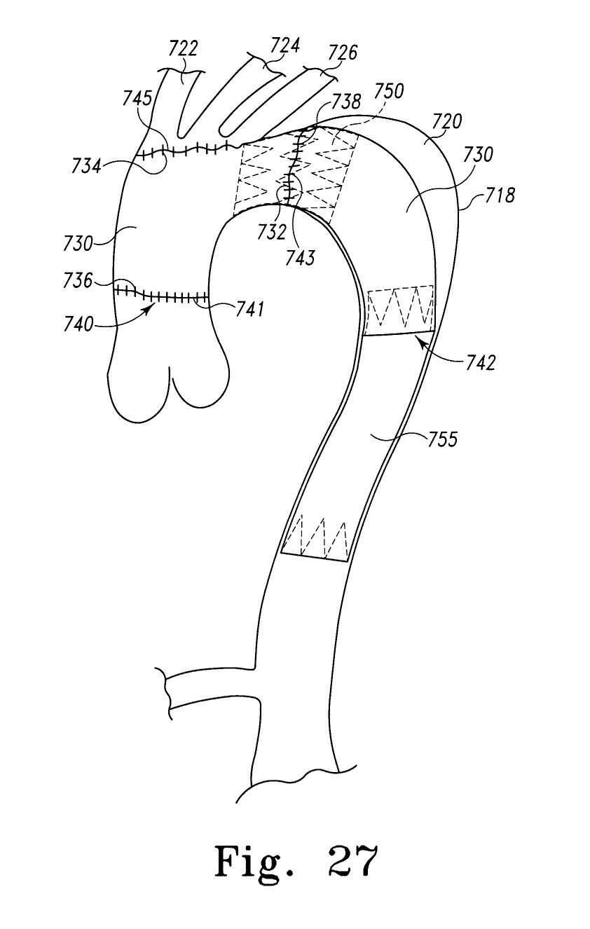

Next, the introducer 600 may be withdrawn from the vessel. At this stage, additional stent grafts may be provided and intraluminally delivered and deployed in an overlapping relationship with other vessel junctions 641, 645. To complete the procedure, the incision 680 is closed, for example by sutures, as shown in FIG. 26.

FIG. 27 depicts another example of a hybrid or intraoperative procedure using one or more of the devices and/or methods taught in U.S. Pat. No. 6,773,457, entitled “Aortic Graft Device,” which is herein incorporated by reference. In this example, the aortic arch has been resected or removed, as described above. The resection exposes a proximal end opening 732 in the descending aorta 718, a proximal end opening 734 adjacent branch vessels 722, 724, 726, and a distal end opening 736 in the ascending aorta. A graft 730 is provided and has a proximal end 740 and a distal end 742. The proximal end 740 is joined to the distal end opening 736 to form vessel junction 741. An opening or fenestration is provided in the wall of the graft 730 and is joined to the proximal end opening 734 to form vessel junction 745. A medial rim area 738 of the graft 730 is joined to proximal end opening 732 to form junction 743 and the distal end 742 of the graft extends distally within the descending aorta 718 and within the aneurysm 720. A stent graft 755 may be intraluminally delivered and deployed at the distal end of the graft 730 to secure the distal end to the aorta. The junctions 741, 743, 745 may be secured by various suturing devices or techniques, as described above.

One or more additional stent grafts may be provided and intraluminally delivered and deployed to overlap and seal one or more of the junctions 741, 743, 745. In FIG. 27, a stent graft 750 is disposed at junction 743 so that the proximal end of the stent graft 750 is disposed within graft 730 and the distal end of the stent graft is disposed within the aorta. In this example, the distal end of the stent graft 750 is also disposed within the graft 730. The stent graft 750 overlaps and seals vessel junction 743.

Intraluminal access for delivering and deploying the stent grafts 750, 755 may be provided through an incision (not shown) in a proximate vessel, such as the graft 730 or the aorta. Alternatively, access may be provided through a remote vessel, as described above.

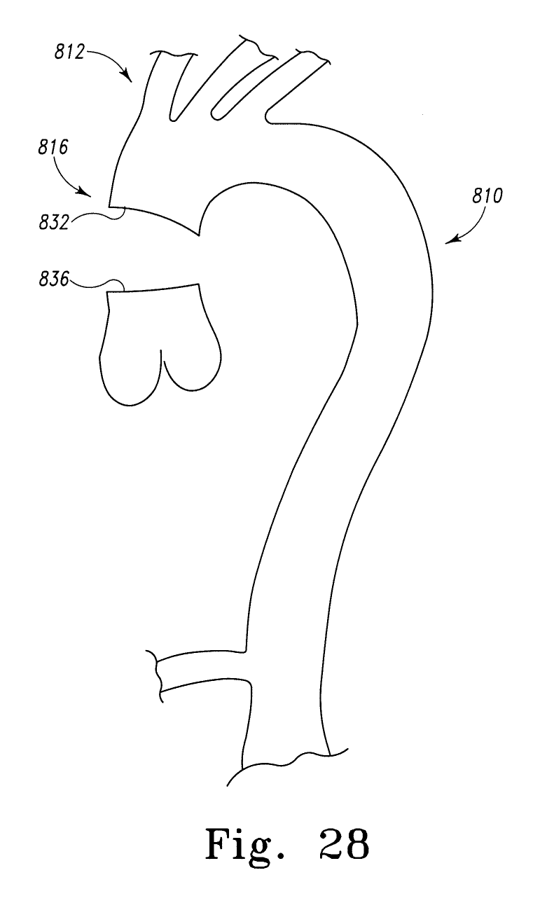

Another exemplary intraoperative method will now be described with reference to FIGS. 28-30, which depict a repair of the ascending aorta. The method may be particularly advantageous for repairs of acute dissections of the distal ascending aorta, although the method could be used to repair other damaged or diseased portions of the aorta or other body vessels.

FIG. 28 shows a schematic view of a region of the aorta 810 including the aortic arch 812. A portion of the ascending aorta 816 is resected and exposes a proximal end opening 832 and a distal end opening 836. Often, in repairs of damaged or diseased body vessels, extensive damage or disease may cause the vessels to lose their elasticity, harden, and to become fragile. In many cases, the body vessel may lack sufficient integrity to adequately support and retain a suture. This presents challenges, where the surgeon wishes to repair the vessel using traditional surgical techniques. In these and other cases, an intraluminal prosthesis may be provided, and an intraoperative procedure performed, to reinforce and repair the body vessel.

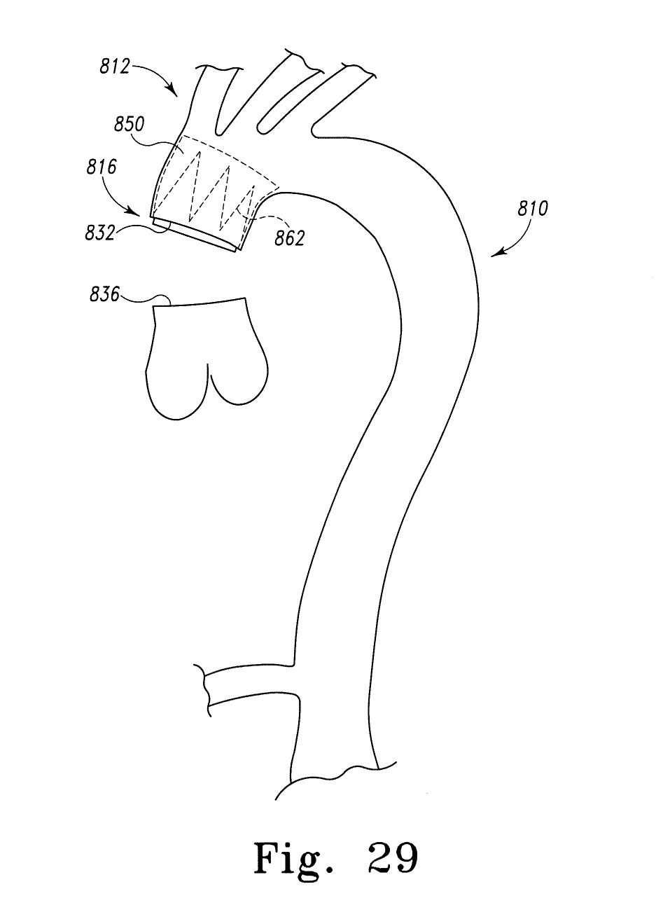

For example, as shown in FIG. 29, a stent graft 850 may be delivered and deployed within the ascending aorta 816 so that the proximal end of the stent graft is disposed adjacent the proximal end opening 832. The stent graft 850 relines and reinforces the ascending aorta 816 and provides additional structure for suturing the proximal end opening 832. The proximal end of the stent graft 850 may be disposed inwardly or outwardly of the proximal end opening 832 and overlap the opening by a short distance. For example, the proximal end of the stent graft 850 may extend outwardly of the proximal end opening 832 by a distance of 0.5 cm. or less, 1 cm. or less, or 2 cm. or less. The stent graft 850 may overlap the vessel opening by other suitable distances, as necessary.

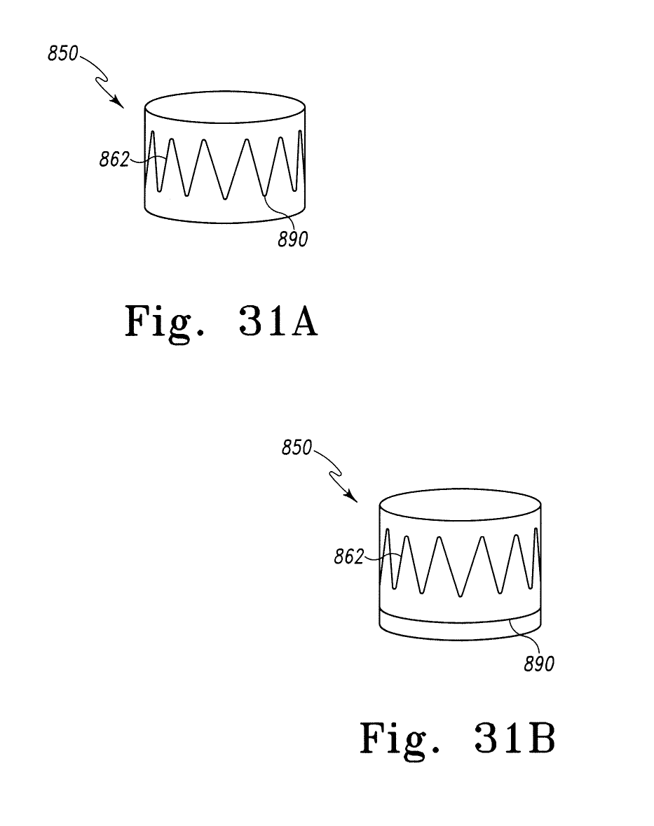

The stent graft 850 comprises one or more stents 862. In the example shown in FIG. 28, the stent graft 850 comprises a single Z-stent 862 and may have a length of approximately 20 mm. Other examples of stent grafts that may be used in the present method are depicted in FIGS. 31A and 31B. In other examples, the stent graft 850 may comprise one or more stents and have a length of less than or greater than 20 mm. The stent graft 850 may comprise barbs or hooks (not shown) that may help anchor the stent graft in place.

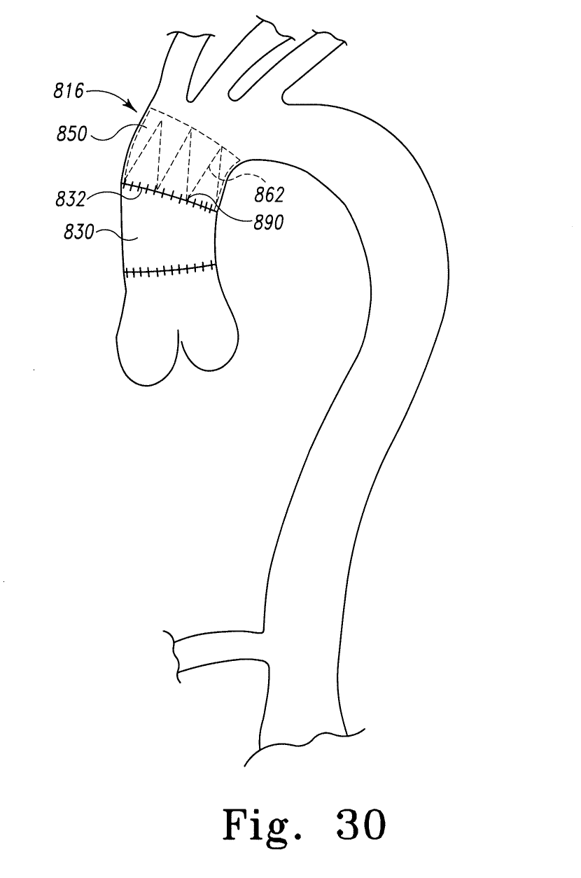

Next, as shown in FIG. 30, a graft 830 may be provided. The proximal end of the graft 830 may be joined to the distal end opening 836 and the distal end of the graft may be joined to the proximal end opening 832, for example, by suturing. In the example shown in FIG. 30, the distal end of the graft 830 is joined with the proximal end opening 832 by suturing the graft 830 to the stent graft 850. The distal end of the graft 830 may also be sutured to the aorta. A suture ring 890 may be provided in the stent graft 850 to enhance the connection between the graft 830 and the stent graft 850. For example, the graft 830 may be sutured to the stent graft 850 via the stent 862. Thus, the stent 862 may act as a suture ring 890 and the sutures may pass around the apices of the stent. In another example, as shown in FIG. 31B, the suture ring 890 may comprise an annular ring structure 892 disposed adjacent an end the stent graft 850. The annular ring 892 may comprise, for example, a metal such as stainless steel or nitinol, or a plastic such as semi-rigid or rigid silicone, urethane, or the like.

Throughout this specification various indications have been given as to preferred and alternative embodiments of the invention. However, it should be understood that the invention is not limited to any one of these. It is therefore intended that the foregoing detailed description be regarded as illustrative rather than limiting, and that it be understood that it is the appended claims, including all equivalents, that are intended to define the spirit and scope of this invention.

1. An intraoperative anastomosis method comprising:
providing an intraluminal prosthesis having a first end and a second end;
suturing a first vessel to a second vessel to form a vessel junction;
intraluminally delivering the intraluminal prosthesis so that the first end of the prosthesis is disposed within the first vessel and the second end of the prosthesis is disposed within the second vessel; and
deploying the intraluminal prosthesis so that the prosthesis overlaps and reinforces the vessel junction;
wherein the vessel suturing, prosthesis delivering, and prosthesis deploying steps are performed in a single operation.

2. The method according to claim 1, wherein one of the first vessel and the second vessel is a body vessel and the other of the second vessel and the first vessel is a prosthetic vessel.

3. The method according to claim 1, wherein both the first vessel and the second vessel are body vessels.

4. The method according to claim 1, wherein both the first vessel and the second vessel are prosthetic vessels.

5. The method of claim 1, further comprising providing intraluminal access for the prosthesis through an incision in one of the first vessel and the second vessel.

6. The method according to claim 1, wherein the prosthesis comprises a graft and at least one stent disposed on an inside surface and/or an outside surface of the graft.

7. The method according to claim 6, wherein the prosthesis comprises at least one hook or barb extending from the at least one stent.

8. The method according to claim 1, wherein the deploying step comprises deploying the stent graft so that at least one stent overlaps the vessel junction.

9. The method of claim 1, wherein the first vessel comprises the aorta and the second vessel comprises a tapered tubular graft having a distal end and a proximal end, the method further comprising the steps of:
placing a distal portion of the tapered tubular graft inside the aorta; and
suturing the proximal end of the graft in place.

10. The method of claim 9, further comprising the steps of:
providing at least one stent attached to the tapered tubular graft at a site adjacent the distal end of the graft;
loading the tapered tubular graft into an introducer;
inserting the introducer through an incision in the aorta; and
deploying the tapered tubular graft inside the aorta.

11. The method of claim 9, wherein suturing the proximal end of the graft in place comprises suturing the proximal end of the graft to the aorta.

12. The method of claim 9, further comprising inserting the tapered tubular graft through an incision in the aorta.

13. The method of claim 9, further comprising inserting the stent graft through an incision in the aorta or through an incision in the tapered tubular graft.

14. The method according to claim 1, wherein:
the first vessel comprises the aorta;
the second vessel comprises a tapered tubular graft having a distal end and a proximal end;
the intraluminal prosthesis comprises a graft and at least one stent disposed on an inside surface and/or an outside surface of the graft and at least one hook or barb extending from the at least one stent;
the deploying step comprises deploying the intraluminal prosthesis so at least one stent overlaps the vessel junction; and
the method further comprises:
providing at least one stent attached to the tapered tubular graft at a site adjacent the distal end of the graft;
loading the graft into an introducer;
inserting the introducer through an incision in the aorta;
placing a distal portion of the tapered tubular graft inside the aorta;
deploying the tapered tubular graft inside the aorta;
suturing the proximal end of the graft to the aorta; and
providing intraluminal access for the intraluminal prosthesis through an incision in the aorta or an incision in the tapered tubular graft.

15. An intraoperative anastomosis method comprising the steps of:
providing an intraluminal prosthesis;
intraluminally delivering and deploying the intraluminal prosthesis within a first vessel so that a first end of the intraluminal prosthesis is disposed adjacent an opening in the first vessel;
joining the first vessel to a second vessel by suturing the first end of the intraluminal prosthesis to an opening in the second vessel.

16. The method of claim 15, wherein the intraluminal prosthesis comprises a graft and at least one stent attached to the graft.

17. The method of claim 16, wherein the prosthesis comprises at least one hook or barb extending from the at least one stent.

18. The method of claim 15, wherein the intraluminal prosthesis comprises a suture ring disposed adjacent the first end, and wherein the suturing step comprises the step of suturing the second vessel opening to the suture ring.

19. The method of claim 15, wherein the first vessel comprises the distal ascending aorta and the second vessel comprises a second graft.

20. The method of claim 15, wherein:
the intraluminal prosthesis comprises:
a graft;
at least one stent attached to the graft
at least one hook or barb extending from the at least one stent; and
a suture ring disposed adjacent the first end of the graft;
the first vessel comprises the distal ascending aorta and the second vessel comprises a second graft; and
the suturing step comprises the step of suturing the second vessel opening to the suture ring.
